(19) United States
(12) Patent Application Publication
(10) Pub. No.: US 2009/0170891 A1
(43) Pub. Date: Jul. 2, 2009

(54) Niacin Receptor Agonists, Compositions Containing Such Compounds and Methods of Treatment

(76) Inventors: Steven L. Colletti, Princeton Junction, NJ (US); James R. Tata, Westfield, NJ (US); Weichun Chen, Livingston, NJ (US); Richard T. Beresis, San Francisco, CA (US); Fa-Xiang Ding, Staten Island, NY (US); Darby Rye Schmidt, Clark, NJ (US); Hong Shen, West Windsor, NJ (US); Subharekha Raghavan, Teaneck, NJ (US)

Correspondence Address: MERCK AND CO., INC, P O BOX 2000, RAHWAY, NJ, 07065-0907, US

(21) Appl. No.: 12/226,050

(22) PCT Filed: Apr. 6, 2007
(86) PCT No.: PCT/US07/08584

§ 371 Date: Oct. 6, 2008

Related U.S. Application Data
(60) Provisional application No. 60/791,019, filed on Apr. 11, 2006.

(51) Int. Cl. A61K 31/4375; C07D 333/36; A61K 31/381; C07D 413/04; A61K 31/444; C07D 333/38; A61P 3/10

(52) U.S. Cl. 514/292; 549/69; 514/447; 546/269.4; 514/340; 549/61

(57) ABSTRACT

The present invention encompasses compounds of Formula I:

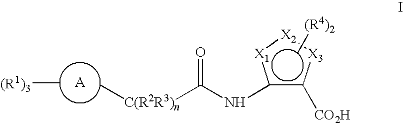

as well as pharmaceutically acceptable salts and hydrates thereof, that are useful for treating atherosclerosis, dyslipidemias and the like. Pharmaceutical compositions and methods of use are also included.

### BACKGROUND OF THE INVENTION

The present invention relates to heterocyclic acid compounds, their derivatives, compositions containing such compounds and methods of treatment or prevention in a mammal relating to dyslipidemias. Dyslipidemia is a condition wherein serum lipids are abnormal. Elevated cholesterol and low levels of high density lipoprotein (HDL) are independent risk factors for atherosclerosis associated with a greater-than-normal risk of atherosclerosis and cardiovascular disease. Factors known to affect serum cholesterol include genetic predisposition, diet, body weight, degree of physical activity, age and gender. While cholesterol in normal amounts is a vital building block for cell membranes and essential organic molecules such as steroids and bile acids, cholesterol in excess is known to contribute to cardiovascular disease. For example, cholesterol, through its relationship with foam cells, is a primary component of plaque which collects in coronary arteries, resulting in the cardiovascular disease termed atherosclerosis.

Traditional therapies for reducing cholesterol include medications such as statins (which reduce production of cholesterol by the body). More recently, the value of nutrition and nutritional supplements in reducing blood cholesterol has received significant attention. For example, dietary compounds such as soluble fiber, vitamin E, soy, garlic, omega-3 fatty acids, and niacin have all received significant attention and research funding.

Niacin or nicotinic acid (pyridine-3-carboxylic acid) is a drug that reduces coronary events in clinical trials. It is commonly known for its effect in elevating serum levels of high density lipoproteins (HDL). Importantly, niacin also has a beneficial effect on other lipid profiles. Specifically, it reduces low density lipoproteins (LDL), very low density lipoproteins (VLDL), and triglycerides (TG). However, the clinical use of nicotinic acid is limited by a number of adverse side-effects including cutaneous vasodilation, sometimes called flushing.

Despite the attention focused on traditional and alternative means for controlling serum cholesterol, serum triglycerides, and the like, a significant portion of the population has total cholesterol levels greater than about 200 mg/dL, and are thus candidates for dyslipidemia therapy. There thus remains a need in the art for compounds, compositions and alternative methods of reducing total cholesterol, serum triglycerides, and the like, and raising HDL.

The present invention relates to compounds that have been discovered to have effects in modifying serum lipid levels.

The invention thus provides compositions for effecting reduction in total cholesterol and triglyceride concentrations and raising HDL, in accordance with the methods described.

Consequently one object of the present invention is to provide a nicotinic acid receptor agonist that can be used to treat dyslipidemias, atherosclerosis, diabetes, metabolic syndrome and related conditions while minimizing the adverse effects that are associated with niacin treatment.

Yet another object is to provide a pharmaceutical composition for oral use.

These and other objects will be apparent from the description provided herein.

### SUMMARY OF THE INVENTION

A compound represented by formula I:

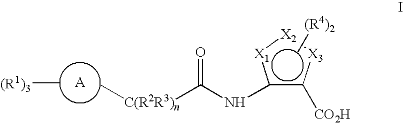

or a pharmaceutically acceptable salt or solvate thereof is disclosed wherein:

one of X¹, X² and X³ represents a sulfur atom, and the other two represent carbon or nitrogen atoms;

ring A represents a 6-10 membered aryl, or a 5-13 membered heteroaryl or partially aromatic heterocyclic group, said heteroaryl and partially aromatic heterocyclic group containing at least one heteroatom selected from O, S, S(O), S(O)₂ and N, and optionally containing 1 other heteroatom selected from O and S, and optionally containing 1-3 additional N atoms, with up to 5 heteroatoms being present;

each R² and R³ is independently H, C₁₋₃alkyl, haloC₁₋₃alkyl, OC₁₋₃alkyl, haloC₁₋₃alkoxy, OH or F;

n represents an integer of from 2 to 4;

each R⁴ is H or is independently selected from halo, SC₁₋₄alkyl, CN, C₁₋₄alkyl, C₁₋₄alkoxy, haloC₁₋₄alkyl and haloC₁₋₄alkoxy;

and each R¹ is H or is independently selected from the group consisting of:

a) halo, OH, CO₂H, CN, NH₂, S(O)₀₋₂R^{e}, C(O)R^{e}, OC(O)R^{e} and CO₂R^{e}, wherein R^{e} is C₁₋₄alkyl or phenyl, each being optionally substituted with 1-3 groups, 1-3 of which are halo or C₁₋₃alkyl, and 1-2 of which are selected from OC₁₋₃alkyl, haloC₁₋₃alkyl, haloC₁₋₃alkoxy, OH, NH₂ and NHC₁₋₃alkyl;

b) C₁₋₆ alkyl and OC₁₋₆alkyl, said C₁₋₆alkyl and alkyl portion of OC₁₋₆alkyl being optionally substituted with 1-3 groups, 1-3 of which are halo and 1-2 of which are selected from: OH, CO₂H, CO₂C₁₋₄alkyl, CO₂C₁₋₄haloalkyl, OCO₂C₁₋₄alkyl, NH₂, NHC₁₋₄alkyl, N(C₁₋₄alkyl)₂, Hetcy and CN;

c) NHC₁₋₄alkyl and N(C₁₋₄alkyl)₂, the alkyl portions of which are optionally substituted as set forth in (b) above;

d) C(O)NH₂, C(O)NHC₁₋₄alkyl, C(O)N(C₁₋₄alkyl)₂, C(O)Hetcy, C(O)NHOC₁₋₄alkyl and C(O)N(C₁₋₄alkyl)(OC₁₋₄alkyl), the alkyl portions of which are optionally substituted as set forth in (b) above;

e) NR′C(O)R″, NR′SO₂R″, NR′CO₂R″ and NR′C(O)NR″R′″ wherein:

R′ represents H, C₁₋₃alkyl or haloC₁₋₃alkyl,

R″ represents (a) C₁₋₈alkyl optionally substituted with 1-4 groups, 0-4 of which are halo, and 0-1 of which are selected from the group consisting of: OC₁₋₆alkyl, OH, CO₂H, CO₂C₁₋₄alkyl, CO₂C₁₋₄haloalkyl, NH₂, NHC₁₋₄alkyl, N(C₁₋₄alkyl)₂, CN, Hetcy, Aryl and HAR,
said Hetcy, Aryl and HAR being further optionally substituted with 1-3 halo, C₁₋₄alkyl, C₁₋₄alkoxy, haloC₁₋₄alkyl or haloC₁₋₄alkoxy groups;
   (b) Hetcy, Aryl or HAR, each being optionally substituted with 1-3 members selected from the group consisting of: halo, C₁₋₄alkyl, C₁₋₄alkoxy, haloC₁₋₄alkyl and haloC₁₋₄alkoxy groups;

and R′″ representing H or R″;

f) phenyl or a 5-6 membered heteroaryl or a Hetcy group attached at any available ring atom and each being optionally substituted with 1-3 groups, 1-3 of which are selected from halo, C₁₋₃alkyl and haloC₁₋₃alkyl groups, and 1-2 of which are selected from OC₁₋₃alkyl and haloOC₁₋₃alkyl groups, and 0-1 of which is selected from the group consisting of:
i) OH; CO₂H; CN; NH₂ and S(O)₀₋₂R^{e} wherein R^{e} is as described above;
ii) NHC₁₋₄alkyl and N(C₁₋₄alkyl)₂, the alkyl portions of which are optionally substituted with 1-3 groups, 1-3 of which are halo and 1-2 of which are selected from: OH, CO₂H, CO₂C₁₋₄alkyl, CO₂C₁₋₄haloalkyl, NH₂, NHC₁₋₄alkyl, N(C₁₋₄alkyl)₂ and CN;
iii) C(O)NH₂, C(O)NHC₁₋₄alkyl, C(O)N(C₁₋₄alkyl)₂, C(O)NHOC₁₋₄alkyl and C(O)N(C₁₋₄alkyl)(OC₁₋₄alkyl), the alkyl portions of which are optionally substituted as set forth in b) above; and
iv) NR′C(O)R″, NR′SO₂R″, NR′CO₂R″ and NR′C(O)NR″R′″ wherein R′, R″ and R′″ are as described above.

### DETAILED DESCRIPTION OF THE INVENTION

The invention is described herein in detail using the terms defined below unless otherwise specified.

“Alkyl”, as well as other groups having the prefix “alk”, such as alkoxy, alkanoyl and the like, means carbon chains which may be linear, branched, or cyclic, or combinations thereof, containing the indicated number of carbon atoms. If no number is specified, 1-6 carbon atoms are intended for linear and 3-7 carbon atoms for branched alkyl groups. Examples of alkyl groups include methyl, ethyl, propyl, isopropyl, butyl, sec- and tert-butyl, pentyl, hexyl, heptyl, octyl, nonyl and the like. Cycloalkyl is a subset of alkyl; if no number of atoms is specified, 3-7 carbon atoms are intended, forming 1-3 carbocyclic rings that are fused. “Cycloalkyl” also includes monocyclic rings fused to an aryl group in which the point of attachment is on the non-aromatic portion. Examples of cycloalkyl include cyclopropyl, cyclobutyl, cyclopentyl, cyclohexyl, cycloheptyl, tetrahydronaphthyl, decahydronaphthyl, indanyl and the like.

“Alkenyl” means carbon chains which contain at least one carbon-carbon double bond, and which may be linear or branched or combinations thereof. Examples of alkenyl include vinyl, allyl, isopropenyl, pentenyl, hexenyl, heptenyl, 1-propenyl, 2-butenyl, 2-methyl-2-butenyl, and the like.

“Alkynyl” means carbon chains which contain at least one carbon-carbon triple bond, and which may be linear or branched or combinations thereof. Examples of alkynyl include ethynyl, propargyl, 3-methyl-1-pentynyl, 2-heptynyl and the like.

“Aryl” (Ar) means mono- and bicyclic aromatic rings containing 6-10 carbon atoms. Examples of aryl include phenyl, naphthyl, indenyl and the like.

“Heteroaryl” (HAR) unless otherwise specified, means mono-, bicyclic and tricyclic aromatic ring systems containing at least one heteroatom selected from O, S, S(O), SO₂ and N, with each ring containing 5 to 6 atoms. HAR groups may contain from 5-14, preferably 5-13 atoms. Examples include, but are not limited to, pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, pyrazinyl, benzoxazolyl, benzothiazolyl, benzimidazolyl, benzofuranyl, benzothiophenyl, benzopyrazolyl, benzotriazolyl, furo(2,3-b)pyridyl, benzoxazinyl, tetrahydrohydroquinolinyl, tetrahydroisoquinolinyl, quinolyl, isoquinolyl, indolyl, dihydroindolyl, quinoxalinyl, quinazolinyl, naphthyridinyl, pteridinyl, 2,3-dihydrofuro(2,3-b)pyridyl and the like. Heteroaryl also includes aromatic carbocyclic or heterocyclic groups fused to heterocycles that are non-aromatic or partially aromatic, and optionally containing a carbonyl. Examples of additional heteroaryl groups include indolinyl, dihydrobenzofuranyl, dihydrobenzothiophenyl, dihydrobenzoxazolyl, and aromatic heterocyclic groups fused to cycloalkyl rings. Examples also include the following:

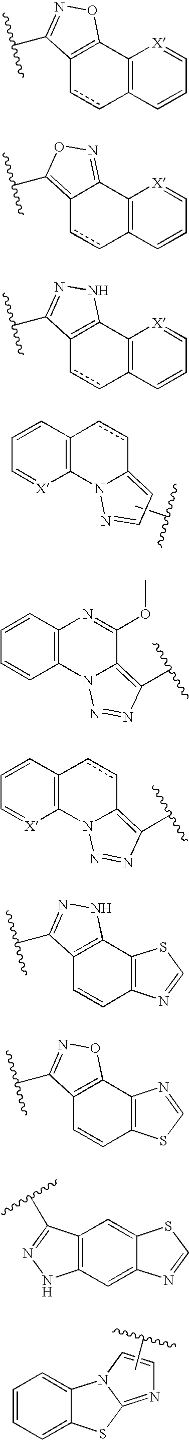

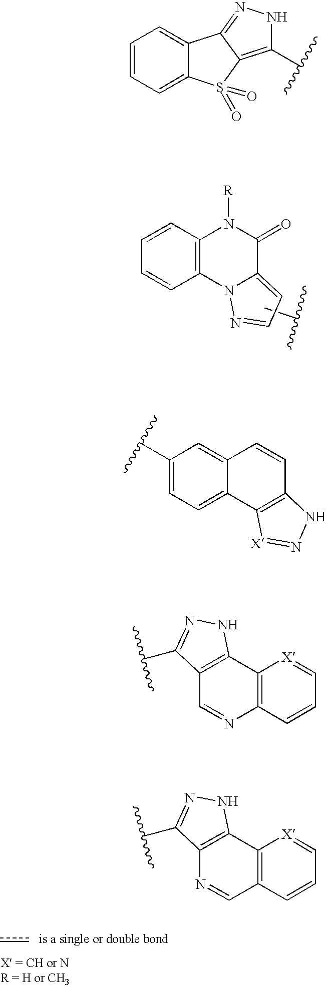

Heteroaryl also includes such groups in charged form, e.g., pyridinium.

“Heterocyclyl” (Hetcy) unless otherwise specified, means mono- and bicyclic saturated rings and ring systems containing at least one heteroatom selected from N, S and O, each of said ring having from 3 to 10 atoms in which the point of attachment may be carbon or nitrogen. Examples of “heterocyclyl” include, but are not limited to, azetidinyl, pyrrolidinyl, piperidinyl, piperazinyl, imidazolidinyl, tetrahydrofuranyl, 1,4-dioxanyl, morpholinyl, thiomorpholinyl, tetrahydrothienyl and the like. Heterocycles can also exist in tautomeric forms, e.g., 2- and 4-pyridones. Heterocycles moreover includes such moieties in charged form, e.g., piperidinium.

“Halogen” (Halo) includes fluorine, chlorine, bromine and iodine.

The phrase “in the absence of substantial flushing” refers to the side effect that is often seen when nicotinic acid is administered in therapeutic amounts. The flushing effect of nicotinic acid usually becomes less frequent and less severe as the patient develops tolerance to the drug at therapeutic doses, but the flushing effect still occurs to some extent and can be transient. Thus, “in the absence of substantial flushing” refers to the reduced severity of flushing when it occurs, or fewer flushing events than would otherwise occur. Preferably, the incidence of flushing (relative to niacin) is reduced by at least about a third, more preferably the incidence is reduced by half, and most preferably, the flushing incidence is reduced by about two thirds or more. Likewise, the severity (relative to niacin) is preferably reduced by at least about a third, more preferably by at least half, and most preferably by at least about two thirds. Clearly a one hundred percent reduction in flushing incidence and severity is most preferable, but is not required.

One aspect of the invention relates to a compound represented by formula I:

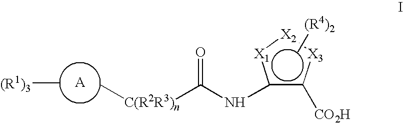

or a pharmaceutically acceptable salt or solvate thereof is disclosed wherein:

one of X¹, X² and X³ represents a sulfur atom, and the other two represent carbon or nitrogen atoms;

ring A represents a 6-10 membered aryl; or a 5-13 membered heteroaryl or partially aromatic heterocyclic group, said heteroaryl and partially aromatic heterocyclic group containing at least one heteroatom selected from O, S, S(O), S(O)₂ and N, and optionally containing 1 other heteroatom selected from O and S, and optionally containing 1-3 additional N atoms, with up to 5 heteroatoms being present;

each R² and R³ is independently H, C₁₋₃alkyl, haloC₁₋₃alkyl, OC₁₋₃alkyl, haloC₁₋₃alkoxy, OH or F;

n represents an integer of from 2 to 4;

each R⁴ is H or is independently selected from halo, SC₁₋₄alkyl, CN, C₁₋₄alkyl, C₁₋₄alkoxy, haloC₁₋₄alkyl and haloC₁₋₄alkoxy;

and each R¹ is H or is independently selected from the group consisting of:

a) halo, OH, CO₂H, CN, NH₂, S(O)₀₋₂R^{e}, C(O)R^{e}, OC(O)R^{e} and CO₂R^{e}, wherein R^{e} is C₁₋₄alkyl or phenyl, each being optionally substituted with 1-3 groups, 1-3 of which are halo or C₁₋₃alkyl, and 1-2 of which are selected from OC₁₋₃alkyl, haloC₁₋₃alkyl, haloC₁₋₃alkoxy, OH, NH₂ and NHC₁₋₃alkyl;

b) C₁₋₆ alkyl and OC₁₋₆alkyl, said C₁₋₆alkyl and alkyl portion of OC₁₋₆alkyl being optionally substituted with 1-3 groups, 1-3 of which are halo and 1-2 of which are selected from: OH, CO₂H, CO₂C₁₋₄alkyl, CO₂C₁₋₄haloalkyl, OCO₂C₁₋₄alkyl, NH₂, NHC₁₋₄alkyl, N(C₁₋₄alkyl)₂, Hetcy and CN;

c) NHC₁₋₄alkyl and N(C₁₋₄alkyl)₂, the alkyl portions of which are optionally substituted as set forth in (b) above;

d) C(O)NH₂, C(O)NHC₁₋₄alkyl, C(O)N(C₁₋₄alkyl)₂, C(O)Hetcy, C(O)NHOC₁₋₄alkyl and C(O)N(C₁₋₄alkyl)(OC₁₋₄alkyl), the alkyl portions of which are optionally substituted as set forth in (b) above;

e) NR′C(O)R″, NR′SO₂R″, NR′CO₂R″ and NR′C(O)NR″R′″ wherein:

R′ represents H, C₁₋₃alkyl or haloC₁₋₃alkyl,

R″ represents (a) C₁₋₈alkyl optionally substituted with 1-4 groups, 0-4 of which are halo, and 0-1 of which are selected from the group consisting of: OC₁₋₆alkyl, OH, CO₂H, CO₂C₁₋₄alkyl, CO₂C₁₋₄haloalkyl, NH₂, NHC₁₋₄alkyl, N(C₁₋₄alkyl)₂, CN, Hetcy, Aryl and HAR,
said Hetcy, Aryl and HAR being further optionally substituted with 1-3 halo, C₁₋₄alkyl, C₁₋₄alkoxy, haloC₁₋₄alkyl or haloC₁₋₄alkoxy groups;
   (b) Hetcy, Aryl or HAR, each being optionally substituted with 1-3 members selected from the group consisting of: halo, C₁₋₄alkyl, C₁₋₄alkoxy, haloC₁₋₄alkyl and haloC₁₋₄alkoxy groups;

and R′″ representing H or R″;

f) phenyl or a 5-6 membered heteroaryl or a Hetcy group attached at any available ring atom and each being optionally substituted with 1-3 groups, 1-3 of which are selected from halo, C₁₋₃alkyl and haloC₁₋₃alkyl groups, and 1-2 of which are selected from OC₁₋₃alkyl and haloOC₁₋₃alkyl groups, and 0-1 of which is selected from the group consisting of:
i) OH; CO₂H; CN; NH₂ and S(O)₀₋₂R^{e} wherein R^{e} is as described above;
ii) NHC₁₋₄alkyl and N(C₁₋₄alkyl)₂, the alkyl portions of which are optionally substituted with 1-3 groups, 1-3 of which are halo and 1-2 of which are selected from: OH, CO₂H, CO₂C₁₋₄alkyl, CO₂C₁₋₄haloalkyl, NH₂, NHC₁₋₄alkyl, N(C₁₋₄alkyl)₂ and CN;
iii) C(O)NH₂, C(O)NHC₁₋₄alkyl, C(O)N(C₁₋₄-alkyl)₂, C(O)NHOC₁₋₄alkyl and C(O)N(C₁₋₄alkyl)(OC₁₋₄alkyl), the alkyl portions of which are optionally substituted as set forth in b) above; and
iv) NR′C(O)R″, NR′SO₂R″, NR′CO₂R″ and NR′C(O)NR″R′″ wherein R′, R″ and R′″ are as described above.

A subset of compounds that is of interest relates to compounds of formula I wherein ring A is a phenyl or naphthyl group, a 5-6 membered monocyclic heteroaryl group, or a 9-13 membered bicyclic or tricyclic heteroaryl group. Within this subset of compounds, all other variables are as defined with respect to formula I.

More particularly, a subset of compounds that is of interest relates to compounds of formula I wherein ring A is selected from the group consisting of: phenyl, naphthyl, isoxazolyl, isothiazolyl, pyrazolyl, pyridyl, oxazolyl, oxadiazolyl, thiadiazolyl, thiazolyl, triazolyl, thienyl, pyrimidyl, benzothiazolyl, or a member selected from the group consisting of:

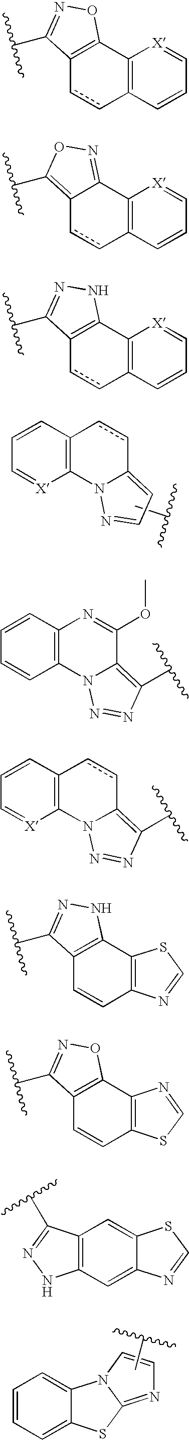

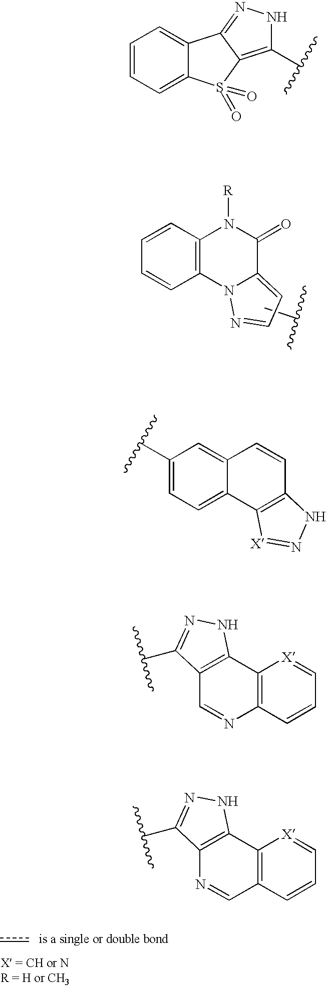

Within this subset of compounds, all other variables are as defined with respect to formula I.

More particularly, a subset of compounds that is of interest relates to compounds of formula I wherein ring A is selected from the group consisting of: phenyl, naphthyl, isoxazolyl, pyrazolyl, oxazolyl, oxadiazolyl, thiazolyl, triazolyl, and benzothiazolyl. Within this subset of compounds, all other variables are as defined with respect to formula I.

Even more particularly, a subset of compounds that is of interest relates to compounds of formula I wherein ring A is selected from the group consisting of: phenyl, naphthyl and oxadiazolyl. Within this subset of compounds, all other variables are as defined with respect to formula I.

Another subset of compounds that is of interest relates to compounds of formula I wherein one of X¹, X² and X³ is S, one is C and one is C or N. Within this subset of compounds, all other variables are as defined with respect to formula I.

More particularly, a subset of compounds that is of interest relates to compounds of formula I wherein one of X¹, X² and X³ is S, and the other two are C. Within this subset of compounds, all other variables are as defined with respect to formula I.

Another subset of compounds that is of interest relates to compounds of formula I wherein each R¹ is H or is selected from the group consisting of:

a) halo, OH, CO₂H, CN, NH₂, S(O)₀₋₂R^{e}, C(O)R^{e}, OC(O)R^{e} and CO₂R^{e}, wherein R^{e} is C₁₋₄alkyl or phenyl, each being optionally substituted with 1-3 groups, 1-3 of which are halo or C₁₋₃alkyl, and 1-2 of which are selected from OC₁₋₃alkyl, haloC₁₋₃alkyl, haloC₁₋₃alkoxy, OH, NH₂ and NHC₁₋₃alkyl;

b) C₁₋₆alkyl and OC₁₋₆alkyl, said C₁₋₆alkyl and alkyl portion of OC₁₋₆alkyl being optionally substituted with 1-3 groups, 1-3 of which are halo and 1-2 of which are selected from: OH, CO₂H, CO₂C₁₋₄alkyl, CO₂C₁₋₄haloalkyl, OCO₂C₁₋₄alkyl, NH₂, NHC₁₋₄alkyl, N(C₁₋₄alkyl)₂, Hetcy and CN; and

c) phenyl or a 5-6 membered heteroaryl or a Hetcy group attached at any available ring atom and each being optionally substituted with 1-3 groups, 1-3 of which are selected from halo, C₁₋₃alkyl and haloC₁₋₃alkyl groups, and 1-2 of which are selected from OC₁₋₃alkyl and haloOC₁₋₃alkyl groups, and 0-1 of which is selected from the group consisting of:
i) OH; CO₂H; CN; NH₂ and S(O)₀₋₂R^{e} wherein R^{e} is as described above;
ii) NHC₁₋₄alkyl and N(C₁₋₄alkyl)₂, the alkyl portions of which are optionally substituted with 1-3 groups, 1-3 of which are halo and 1-2 of which are selected from: OH, CO₂H, CO₂C₁₋₄alkyl, CO₂C₁₋₄haloalkyl, NH₂, NHC₁₋₄alkyl, N(C₁₋₄alkyl)₂ and CN;
iii) C(O)NH₂, C(O)NHC₁₋₄alkyl, C(O)N(C₁₋₄alkyl)₂, C(O)NHOC₁₋₄alkyl and C(O)N(C₁₋₄alkyl)(OC₁₋₄alkyl), the alkyl portions of which are optionally substituted as set forth in b) above; and
iv) NR′C(O)R″, NR′SO₂R″, NR′CO₂R″ and NR′C(O)NR″R′″ wherein R′, R″ and R′″ are as described above with respect for formula I. Within this subset of compounds, all other variables are as defined with respect to formula I.

In particular, another subset of compounds that is of interest relates to compounds of formula I wherein each R¹ is H or is selected from the group consisting of:

a) halo or OH;

b) C₁₋₄alkyl and OC₁₋₄alkyl, each optionally substituted with 1-3 halo groups;

c) phenyl or a 5-6 membered heteroaryl group optionally substituted with 1-3 groups, 1-3 of which are selected from halo, C₁₋₃alkyl and haloC₁₋₃alkyl groups, and 1-2 of which are selected from OC₁₋₃alkyl and haloOC₁₋₃alkyl groups, and 0-1 of which is OH; Within this subset of compounds, all other variables are as defined with respect to formula I.

Even more particularly, an aspect of the invention that is of interest relates to a compound of formula I wherein each R¹ is H or is selected from the group consisting of:

a) halo or OH;

b) C₁₋₃alkyl and OC₁₋₃alkyl;

c) phenyl or pyridyl, each optionally substituted with 1-3 groups, 1-3 of which are selected from halo, 1-2 of which are C₁₋₃alkyl, haloC₁₋₃alkyl, OC₁₋₃alkyl and haloOC₁₋₃alkyl, and 0-1 of which is OH; Within this subset of compounds, all other variables are as defined with respect to formula I.

Another subset of compounds that is of interest relates to a compound of formula I wherein R² and R³ are independently H, C₁₋₃alkyl or haloC₁₋₃alkyl. Within this subset of compounds, all other variables are as defined with respect to formula I.

More particularly, a subset of compounds that is of interest relates to a compound of formula I wherein R² and R³ are independently H or methyl. Within this subset of compounds, all other variables are as defined with respect to formula I.

Another subset of compounds that is of interest relates to a compound of formula I wherein n represents an integer of from 2 or 4. Within this subset of compounds, all other variables are as defined with respect to formula I.

More particularly, a subset of compounds that is of interest relates to a compound of formula I wherein n is 2. Within this subset of compounds, all other variables are as defined with respect to formula I.

More particularly, a subset of compounds that is of interest relates to a compound of formula I wherein n is 4. Within this subset of compounds, all other variables are as defined with respect to formula I.

Another subset of compounds that is of interest relates to a compound of formula I wherein each R⁴ is H or is independently selected from halo, C₁₋₄alkyl, CN and SC₁₋₄alkyl. Within this subset of compounds, all other variables are as defined with respect to formula I.

More particularly, a subset of compounds that is of interest relates to a compound of formula I wherein each R⁴ is H or is independently selected from C₁₋₄alkyl, Cl, CN and SC₁₋₂alkyl. Within this subset of compounds, all other variables are as defined with respect to formula I.

A subset of the invention that is of interest relates to compounds of formula I or a pharmaceutically acceptable salt or solvate thereof wherein:

ring A is a phenyl or naphthyl group, or a 5-6 membered monocyclic heteroaryl group;

one of X¹, X² and X³ is S, one is C and one is C or N;

each R¹ is H or is selected from the group consisting of:

a) halo, OH, CN, NH₂, S(O)₀₋₂R^{e}, C(O)R^{e}, OC(O)R^{e} and CO₂R^{e}, wherein R^{e} is C₁₋₄alkyl or phenyl, each being optionally substituted with 1-3 groups, 1-3 of which are halo or C₁₋₃alkyl, and 1-2 of which are selected from OC₁₋₃alkyl, haloC₁₋₃alkyl, haloC₁₋₃alkoxy, OH, NH₂ and NHC₁₋₃alkyl;

b) C₁₋₆alkyl and OC₁₋₆alkyl, said C₁₋₆alkyl and alkyl portion of OC₁₋₆alkyl being optionally substituted with 1-3 groups, 1-3 of which are halo and 1-2 of which are selected from: OH, CO₂H, CO₂C₁₋₄alkyl, CO₂C₁₋₄haloalkyl, OCO₂C₁₋₄alkyl, NH₂, NHC₁₋₄alkyl, N(C₁₋₄alkyl)₂, Hetcy and CN; and

c) phenyl or a 5-6 membered heteroaryl or a Hetcy group attached at any available ring atom and each being optionally substituted with 1-3 groups, 1-3 of which are selected from halo, C₁₋₃alkyl and haloC₁₋₃alkyl groups, and 1-2 of which are selected from OC₁₋₃alkyl and haloOC₁₋₃alkyl groups, and 0-1 of which is selected from the group consisting of:
i) OH, CN, and NH₂;
ii) C(O)NH₂, C(O)NHC₁₋₄alkyl, C(O)N(C₁₋₄alkyl)₂, C(O)NHOC₁₋₄alkyl and C(O)N(C₁₋₄alkyl)(OC₁₋₄alkyl), the alkyl portions of which are optionally substituted as set forth in b) above; and
iii) NR′C(O)R″, NR′SO₂R″, NR′CO₂R″ and NR′C(O)NR″R′″ wherein R′, R″ and R′″ are as described above with respect to formula I;

R² and R³ are independently H or C₁₋₃alkyl;

n represents the integer 2 or 4; and

R⁴ is H or is independently selected from halo, C₁₋₄alkyl, CN and SC₁₋₄alkyl.

Within this subset of compounds, all other variables are as defined with respect to formula I.

Representative examples of species that are of interest are shown in the table below.

TABLE

| | |
|---|---|
| EXAMPLE 1 | EXAMPLE 2 |
| 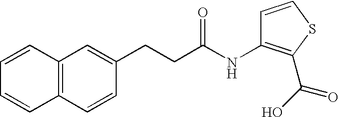 | 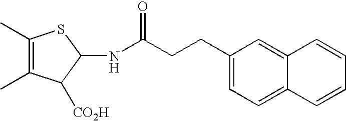 |
| EXAMPLE 3 | EXAMPLE 4 |
| 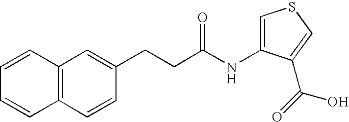 | 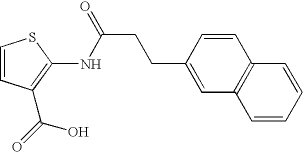 |
| EXAMPLE 5 | EXAMPLE 6 |
| 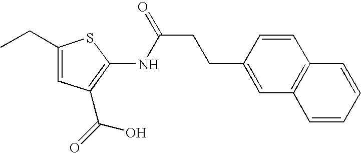 | 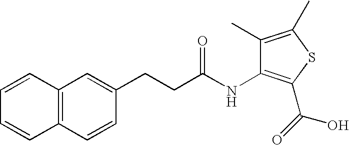 |
| EXAMPLE 7 | EXAMPLE 8 |
| 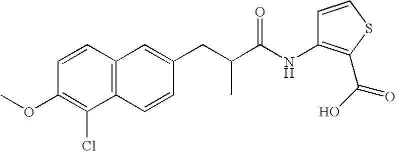 | 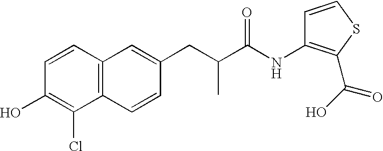 |
| EXAMPLE 9 | EXAMPLE 10 |
| 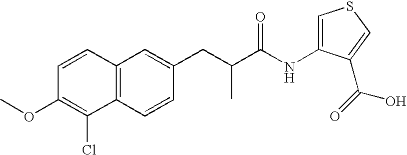 | 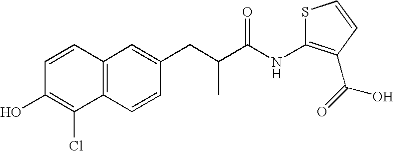 |
| EXAMPLE 11 | EXAMPLE 12 |
| 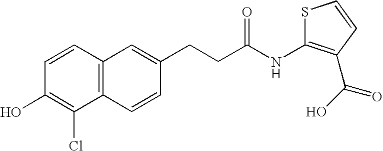 | 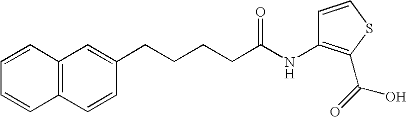 |
| EXAMPLE 13 | EXAMPLE 14 |
| 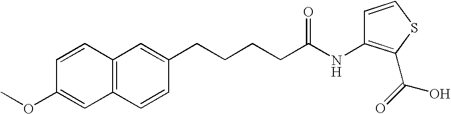 | 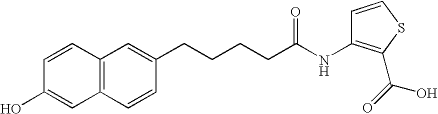 |
| EXAMPLE 15 | EXAMPLE 16 |
| 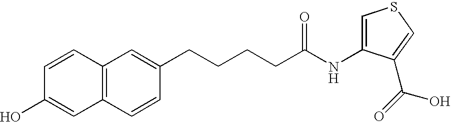 | 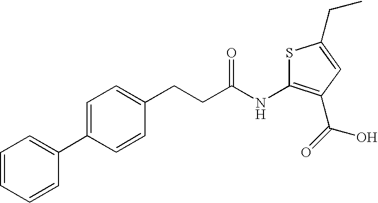 |
| EXAMPLE 17 | EXAMPLE 18 |
| 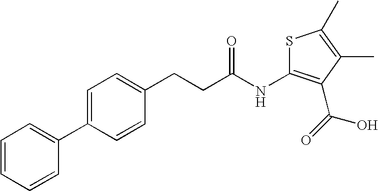 | 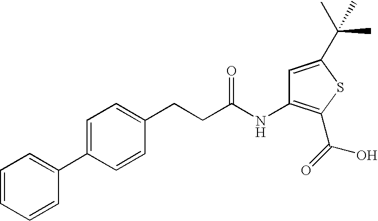 |
| EXAMPLE 19 | EXAMPLE 20 |
| 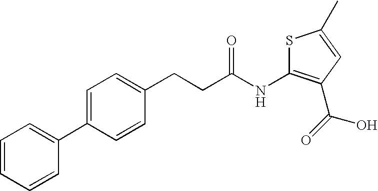 | 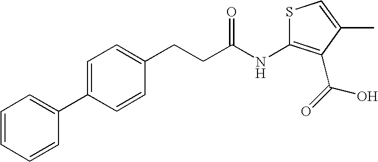 |
| EXAMPLE 21 | EXAMPLE 22 |
| 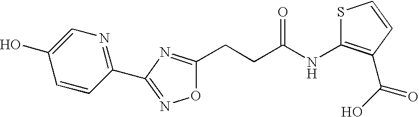 | 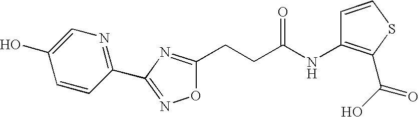 |
| EXAMPLE 23 | EXAMPLE 24 |
| 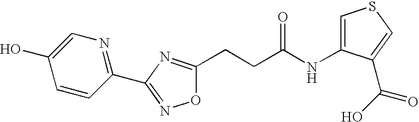 | 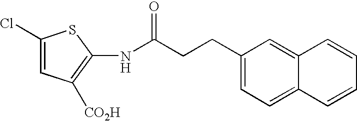 |
| EXAMPLE 25 | EXAMPLE 26 |
| 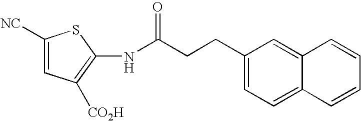 | 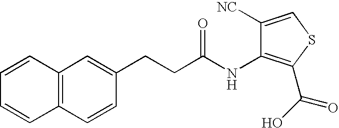 |
| EXAMPLE 27 | EXAMPLE 28 |
| 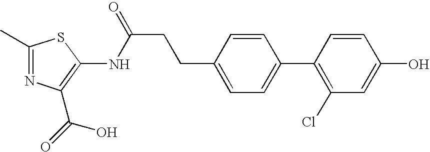 | 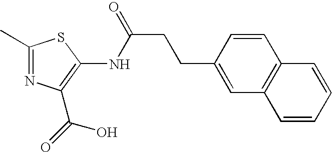 |
| EXAMPLE 29 | EXAMPLE 30 |
| 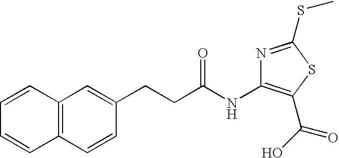 | 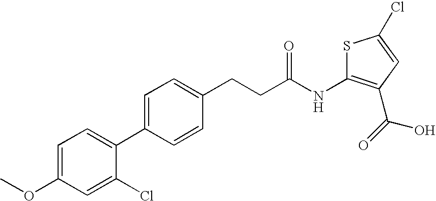 |
| EXAMPLE 31 | EXAMPLE 32 |
| 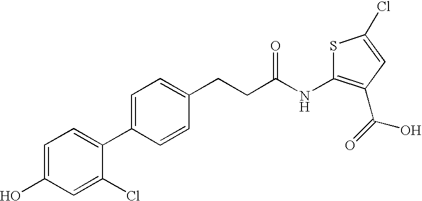 | 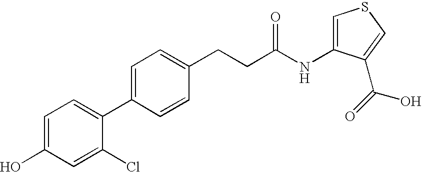 |
| EXAMPLE 33 | EXAMPLE 34 |
| 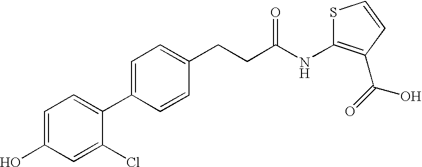 | 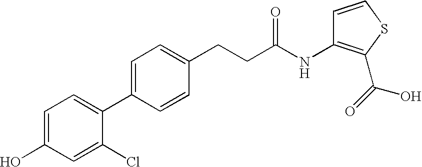 |

EXAMPLE 35

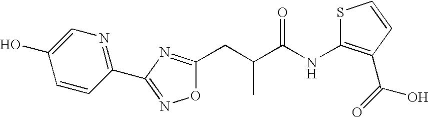

Pharmaceutically acceptable salts and solvates thereof are included as well.

Many of the compounds of formula I contain asymmetric centers and can thus occur as racemates and racemic mixtures, single enantiomers, diastereomeric mixtures and individual diastereomers. All such isomeric forms are included.

Moreover, chiral compounds possessing one stereocenter of general formula I, may be resolved into their enantiomers in the presence of a chiral environment using methods known to those skilled in the art. Chiral compounds possessing more than one stereocenter may be separated into their diastereomers in an achiral environment on the basis of their physical properties using methods known to those skilled in the art. Single diastereomers that are obtained in racemic form may be resolved into their enantiomers as described above.

If desired, racemic mixtures of compounds may be separated so that individual enantiomers are isolated. The separation can be carried out by methods well known in the art, such as the coupling of a racemic mixture of compounds of Formula I to an enantiomerically pure compound to form a diastereomeric mixture, which is then separated into individual diastereomers by standard methods, such as fractional crystallization or chromatography. The coupling reaction is often the formation of salts using an enantiomerically pure acid or base. The diasteromeric derivatives may then be converted to substantially pure enantiomers by cleaving the added chiral residue from the diastereomeric compound.

The racemic mixture of the compounds of Formula I can also be separated directly by chromatographic methods utilizing chiral stationary phases, which methods are well known in the art.

Alternatively, enantiomers of compounds of the general Formula I may be obtained by stereoselective synthesis using optically pure starting materials or reagents.

Some of the compounds described herein exist as tautomers, which have different points of attachment for hydrogen accompanied by one or more double bond shifts. For example, a ketone and its enol form are keto-enol tautomers. Or for example, a 2-hydroxyquinoline can reside in the tautomeric 2-quinolone form. The individual tautomers as well as mixtures thereof are included.

### Dosing Information

The dosages of compounds of formula I or a pharmaceutically acceptable salt or solvate thereof vary within wide limits. The specific dosage regimen and levels for any particular patient will depend upon a variety of factors including the age, body weight, general health, sex, diet, time of administration, route of administration, rate of excretion, drug combination and the severity of the patient's condition. Consideration of these factors is well within the purview of the ordinarily skilled clinician for the purpose of determining the therapeutically effective or prophylactically effective dosage amount needed to prevent, counter, or arrest the progress of the condition. Generally, the compounds will be administered in amounts ranging from as low as about 0.01 mg/day to as high as about 2000 mg/day, in single or divided doses. A representative dosage is about 0.1 mg/day to about 1 g/day. Lower dosages can be used initially, and dosages increased to further minimize any untoward effects. It is expected that the compounds described herein will be administered on a daily basis for a length of time appropriate to treat or prevent the medical condition relevant to the patient, including a course of therapy lasting months, years or the life of the patient.

### Combination Therapy

One or more additional active agents may be administered with the compounds described herein. The additional active agent or agents can be lipid modifying compounds or agents having other pharmaceutical activities, or agents that have both lipid-modifying effects and other pharmaceutical activities. Examples of additional active agents which may be employed include but are not limited to HMG-CoA reductase inhibitors, which include statins in their lactonized or dihydroxy open acid forms and pharmaceutically acceptable salts and esters thereof, including but not limited to lovastatin (see U.S. Pat. No. 4,342,767), simvastatin (see U.S. Pat. No. 4,444,784), dihydroxy open-acid simvastatin, particularly the ammonium or calcium salts thereof, pravastatin, particularly the sodium salt thereof (see U.S. Pat. No. 4,346,227), fluvastatin particularly the sodium salt thereof (see U.S. Pat. No. 5,354,772), atorvastatin, particularly the calcium salt thereof (see U.S. Pat. No. 5,273,995), pitavastatin also referred to as NK-104 (see PCT international publication number WO 97/23200) and rosuvastatin, also known as CRESTOR®; see U.S. Pat. No. 5,260,440); HMG-CoA synthase inhibitors; squalene epoxidase inhibitors; squalene synthetase inhibitors (also known as squalene synthase inhibitors), acyl-coenzyme A: cholesterol acyltransferase (ACAT) inhibitors including selective inhibitors of ACAT-1 or ACAT-2 as well as dual inhibitors of ACAT-1 and -2; microsomal triglyceride transfer protein (MTP) inhibitors; endothelial lipase inhibitors; bile acid sequestrants; LDL receptor inducers; platelet aggregation inhibitors, for example glycoprotein IIb/IIIa fibrinogen receptor antagonists and aspirin; human peroxisome proliferator activated receptor gamma (PPAR-gamma) agonists including the compounds commonly referred to as glitazones for example pioglitazone and rosiglitazone and, including those compounds included within the structural class known as thiazolidine diones as well as those PPAR-gamma agonists outside the thiazolidine dione structural class; PPAR-alpha agonists such as clofibrate, fenofibrate including micronized fenofibrate, and gemfibrozil; PPAR dual alpha/gamma agonists; vitamin B₆ (also known as pyridoxine) and the pharmaceutically acceptable salts thereof such as the HCl salt; vitamin B₁₂ (also known as cyanocobalamin); folic acid or a pharmaceutically acceptable salt or ester thereof such as the sodium salt and the methylglucamine salt; anti-oxidant vitamins such as vitamin C and E and beta carotene; beta-blockers; angiotensin II antagonists such as losartan; angiotensin converting enzyme inhibitors, such as enalapril and captopril; renin inhibitors, calcium channel blockers such as nifedipine and diltiazem; endothelin antagonists; agents that enhance ABCA1 gene expression; cholesteryl ester transfer protein (CETP) inhibiting compounds, 5-lipoxygenase activating protein (FLAP) inhibiting compounds, 5-lipoxygenase (5-LO) inhibiting compounds, farnesoid X receptor (FXR) ligands including both antagonists and agonists; Liver X Receptor (LXR)-alpha ligands, LXR-beta ligands, bisphosphonate compounds such as alendronate sodium; cyclooxygenase-2 inhibitors such as rofecoxib and celecoxib; and compounds that attenuate vascular inflammation.

Cholesterol absorption inhibitors can also be used in the present invention. Such compounds block the movement of cholesterol from the intestinal lumen into enterocytes of the small D intestinal wall, thus reducing serum cholesterol levels. Examples of cholesterol absorption inhibitors are described in U.S. Pat. Nos. 5,846,966, 5,631,365, 5,767,115, 6,133,001, 5,886,171, 5,856,473, 5,756,470, 5,739,321, 5,919,672, and in PCT application Nos. WO 00/63703, WO 00/60107, WO 00/38725, WO 00/34240, WO 00/20623, WO 97/45406, WO 97/16424, WO 97/16455, and WO 95/08532. The most notable cholesterol absorption inhibitor is ezetimibe, also known as 1-(4-fluorophenyl)-3(R)-[3(S)-(4-fluorophenyl)-3-hydroxypropyl)]-4(S)-(4-hydroxyphenyl)-2-azetidinone, described in U.S. Pat. Nos. 5,767,115 and 5,846,966.

Therapeutically effective amounts of cholesterol absorption inhibitors include dosages of from about 0.01 mg/kg to about 30 mg/kg of body weight per day, preferably about 0.1 mg/kg to about 15 mg/kg.

For diabetic patients, the compounds used in the present invention can be administered with conventional diabetic medications. For example, a diabetic patient receiving treatment as described herein may also be taking insulin or an oral antidiabetic medication. One example of an oral antidiabetic medication useful herein is metformin.

In the event that these niacin receptor agonists induce some degree of vasodilation, it is understood that the compounds of formula I may be co-dosed with a vasodilation suppressing agent. Consequently, one aspect of the methods described herein relates to the use of a compound of formula I or a pharmaceutically acceptable salt or solvate thereof in combination with a compound that reduces flushing. Conventional compounds such as aspirin, ibuprofen, naproxen, indomethacin, other NSAIDs, COX-2 selective inhibitors and the like are useful in this regard, at conventional doses. Alternatively, DP antagonists are useful as well. Doses of the DP receptor antagonist and selectivity are such that the DP antagonist selectively modulates the DP receptor without substantially modulating the CRTH2 receptor. In particular, the DP receptor antagonist ideally has an affinity at the DP receptor (i.e., Kᵢ) that is at least about 10 times higher (a numerically lower Kᵢ value) than the affinity at the CRTH2 receptor. Any compound that selectively interacts with DP according to these guidelines is deemed “DP selective”. This is in accordance with US Published Application No. 2004/0229844A1 published on Nov. 18, 2004, incorporated herein by reference.

Dosages for DP antagonists as described herein, that are useful for reducing or preventing the flushing effect in mammalian patients, particularly humans, include dosages ranging from as low as about 0.01 mg/day to as high as about 100 mg/day, administered in single or divided daily doses. Preferably the dosages are from about 0.1 mg/day to as high as about 1.0 g/day, in single or divided daily doses.

Examples of compounds that are particularly useful for selectively antagonizing DP receptors and suppressing the flushing effect include the following:

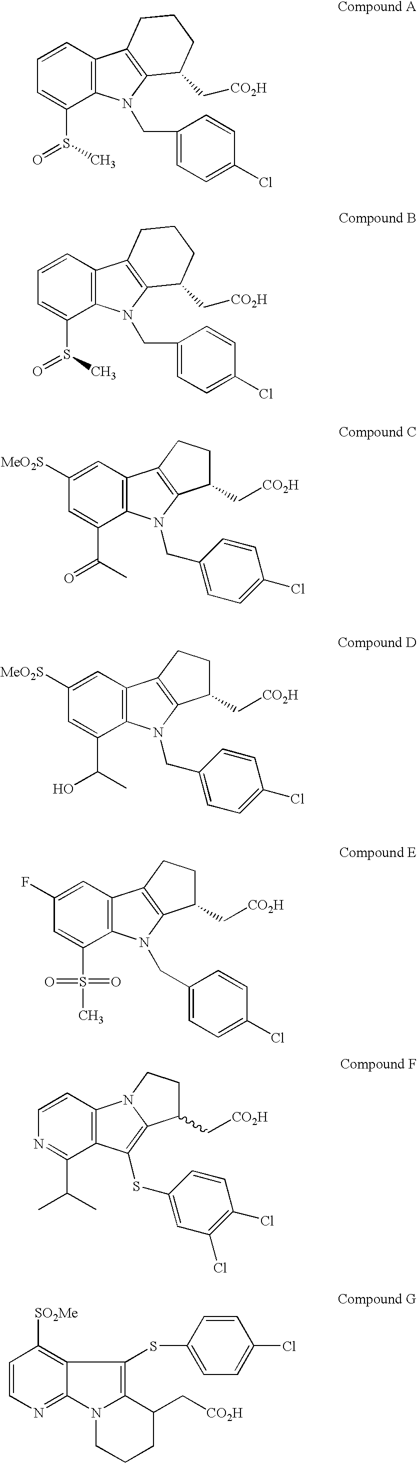

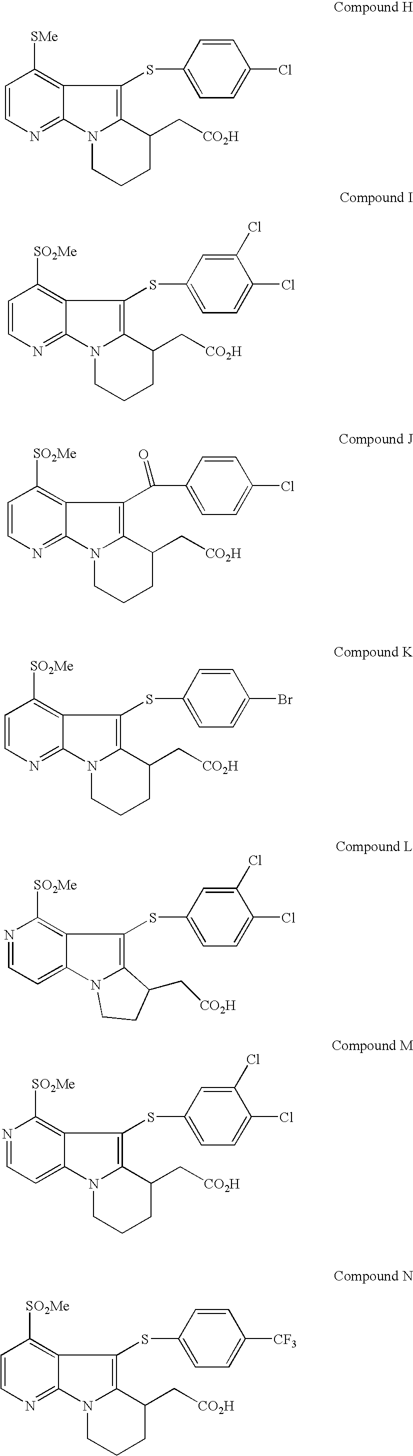

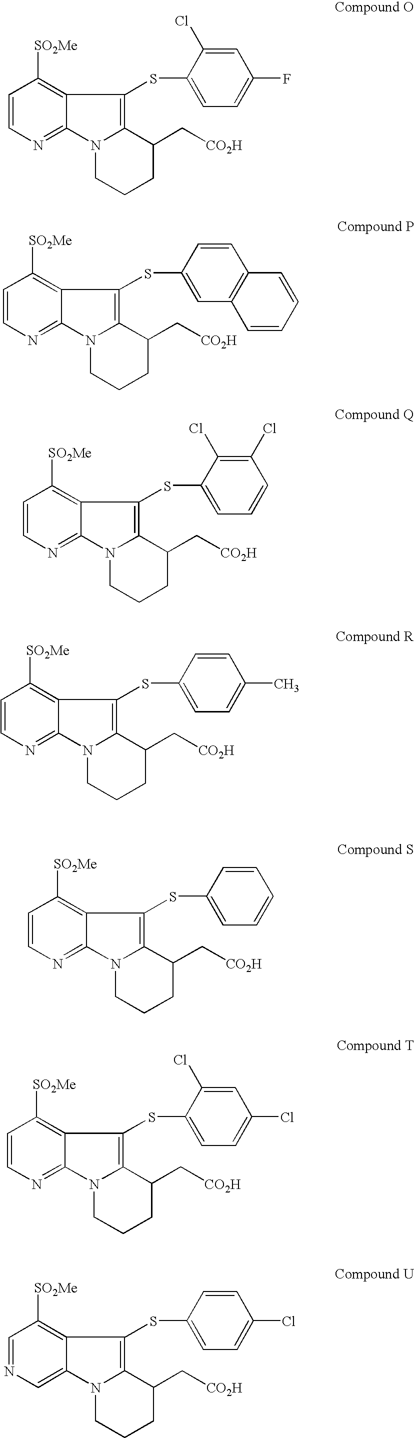

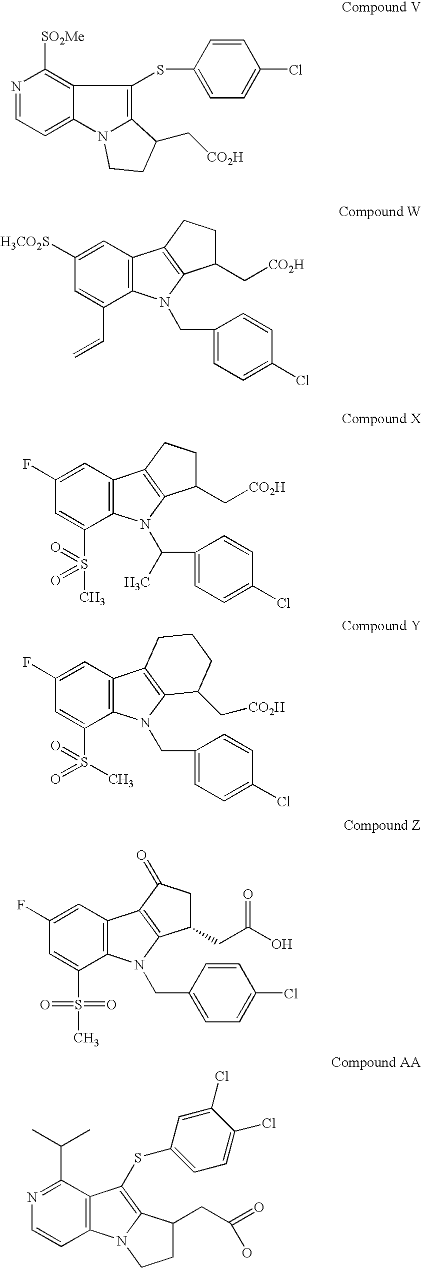

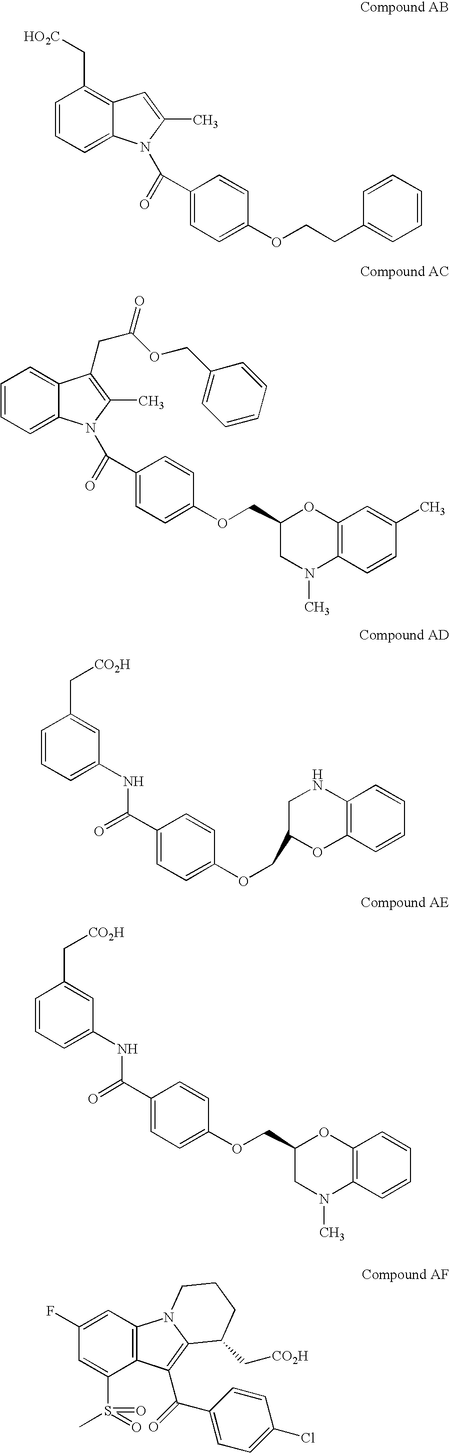

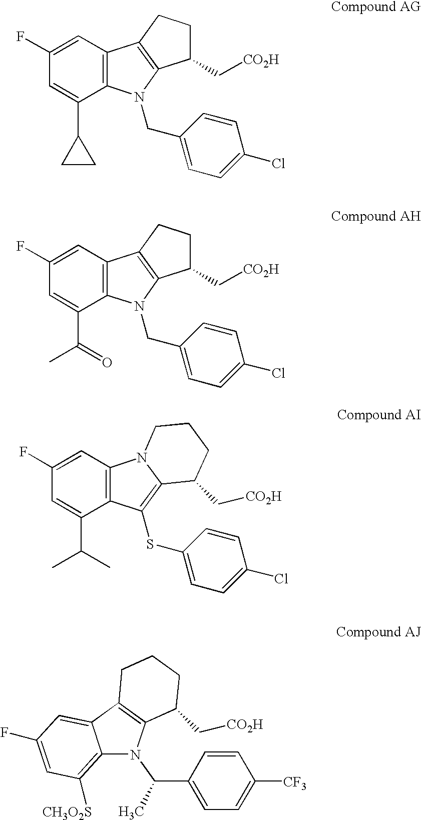

as well as the pharmaceutically acceptable salts and solvates thereof.

The compound of formula I or a pharmaceutically acceptable salt or solvate thereof and the DP antagonist can be administered together or sequentially in single or multiple daily doses, e.g., bid, tid or qid, without departing from the invention. If sustained release is desired, such as a sustained release product showing a release profile that extends beyond 24 hours, dosages may be administered every other day. However, single daily doses are preferred. Likewise, morning or evening dosages can be utilized.

### Salts and Solvates

Salts and solvates of the compounds of formula I are also included in the present invention, and numerous pharmaceutically acceptable salts and solvates of nicotinic acid are useful in this regard. Alkali metal salts, in particular, sodium and potassium, form salts that are useful as described herein. Likewise alkaline earth metals, in particular, calcium and magnesium, form salts that are useful as described herein. Various salts of amines, such as ammonium and substituted ammonium compounds also form salts that are useful as described herein. Similarly, solvated forms of the compounds of formula I are useful within the present invention. Examples include the hemihydrate, mono-, di-, tri- and sesquihydrate.

The heterocyclic acid compounds of the invention also include esters of formula I that are pharmaceutically acceptable, as well as those that are metabolically labile. Metabolically labile esters include C₁₋₄ alkyl esters, preferably the ethyl ester. Many prodrug strategies are known to those skilled in the art. One such strategy involves engineered amino acid anhydrides possessing pendant nucleophiles, such as lysine, which can cyclize upon themselves, liberating the free acid. Similarly, acetone-ketal diesters, which can break down to acetone, an acid and the active acid, can be used.

The compounds used in the present invention can be administered via any conventional route of administration. The preferred route of administration is oral.

### Pharmaceutical Compositions

The pharmaceutical compositions described herein are generally comprised of a compound of formula I or a pharmaceutically acceptable salt or solvate thereof, in combination with a pharmaceutically acceptable carrier.

Examples of suitable oral compositions include tablets, capsules, troches, lozenges, suspensions, dispersible powders or granules, emulsions, syrups and elixirs. Examples of carrier ingredients include diluents, binders, disintegrants, lubricants, sweeteners, flavors, colorants, preservatives, and the like. Examples of diluents include, for example, calcium carbonate, sodium carbonate, lactose, calcium phosphate and sodium phosphate. Examples of granulating and disintegrants include corn starch and alginic acid. Examples of binding agents include starch, gelatin and acacia. Examples of lubricants include magnesium stearate, calcium stearate, stearic acid and talc. The tablets may be uncoated or coated by known techniques. Such coatings may delay disintegration and thus, absorption in the gastrointestinal tract and thereby provide a sustained action over a longer period.

In one embodiment of the invention, a compound of formula I or a pharmaceutically acceptable salt or solvate thereof is combined with another therapeutic agent and the carrier to form a fixed combination product. This fixed combination product may be a tablet or capsule for oral use.

More particularly, in another embodiment of the invention, a compound of formula I or a pharmaceutically acceptable salt or solvate thereof (about 1 to about 1000 mg) and the second therapeutic agent (about 1 to about 500 mg) are combined with the pharmaceutically acceptable carrier, providing a tablet or capsule for oral use.

Sustained release over a longer period of time may be particularly important in the formulation. A time delay material such as glyceryl monostearate or glyceryl distearate may be employed. The dosage form may also be coated by the techniques described in the U.S. Pat. Nos. 4,256,108; 4,166,452 and 4,265,874 to form osmotic therapeutic tablets for controlled release.

Other controlled release technologies are also available and are included herein. Typical ingredients that are useful to slow the release of nicotinic acid in sustained release tablets include various cellulosic compounds, such as methylcellulose, ethylcellulose, propylcellulose, hydroxypropylcellulose, hydroxyethylcellulose, hydroxypropylmethylcellulose, microcrystalline cellulose, starch and the like. Various natural and synthetic materials are also of use in sustained release formulations. Examples include alginic acid and various alginates, polyvinyl pyrrolidone, tragacanth, locust bean gum, guar gum, gelatin, various long chain alcohols, such as cetyl alcohol and beeswax.

Optionally and of even more interest is a tablet as described above, comprised of a compound of formula I or a pharmaceutically acceptable salt or solvate thereof, and further containing an HMG Co-A reductase inhibitor, such as simvastatin or atorvastatin. This particular embodiment optionally contains the DP antagonist as well.

Typical release time frames for sustained release tablets in accordance with the present invention range from about 1 to as long as about 48 hours, preferably about 4 to about 24 hours, and more preferably about 8 to about 16 hours.

Hard gelatin capsules constitute another solid dosage form for oral use. Such capsules similarly include the active ingredients mixed with carrier materials as described above. Soft gelatin capsules include the active ingredients mixed with water-miscible solvents such as propylene glycol, PEG and ethanol, or an oil such as peanut oil, liquid paraffin or olive oil.

Aqueous suspensions are also contemplated as containing the active material in admixture with excipients suitable for the manufacture of aqueous suspensions. Such excipients include suspending agents, for example sodium carboxymethylcellulose, methylcellulose, hydroxypropylmethylcellulose, sodium alginate, polyvinylpyrrolidone, tragacanth and acacia; dispersing or wetting agents, e.g., lecithin; preservatives, e.g., ethyl, or n-propyl para-hydroxybenzoate, colorants, flavors, sweeteners and the like.

Dispersible powders and granules suitable for preparation of an aqueous suspension by the addition of water provide the active ingredients in admixture with a dispersing or wetting agent, suspending agent and one or more preservatives. Suitable dispersing or wetting agents and suspending agents are exemplified by those already mentioned above.

Syrups and elixirs may also be formulated.

More particularly, a pharmaceutical composition that is of interest is a sustained release tablet that is comprised of a compound of formula I or a pharmaceutically acceptable salt or solvate thereof, and a DP receptor antagonist that is selected from the group consisting of compounds A through AJ in combination with a pharmaceutically acceptable carrier.

Yet another pharmaceutical composition that is of more interest is comprised of a compound of formula I or a pharmaceutically acceptable salt or solvate thereof and a DP antagonist compound selected from the group consisting of compounds A, B, D, E, X, AA, AF, AG, AH, AI and AJ, in combination with a pharmaceutically acceptable carrier.

Yet another pharmaceutical composition that is of more particular interest relates to a sustained release tablet that is comprised of a compound of formula I or a pharmaceutically acceptable salt or solvate thereof, a DP receptor antagonist selected from the group consisting of compounds A, B, D, E, X, AA, AF, AG, AH, AI and AJ, and simvastatin or atorvastatin in combination with a pharmaceutically acceptable carrier.

The term “composition”, in addition to encompassing the pharmaceutical compositions described above, also encompasses any product which results, directly or indirectly, from the combination, complexation or aggregation of any two or more of the ingredients, active or excipient, or from dissociation of one or more of the ingredients, or from other types of reactions or interactions of one or more of the ingredients. Accordingly, the pharmaceutical composition of the present invention encompasses any composition made by admixing or otherwise combining the compounds, any additional active ingredient(s), and the pharmaceutically acceptable excipients.

Another aspect of the invention relates to the use of a compound of formula I or a pharmaceutically acceptable salt or solvate thereof and a DP antagonist in the manufacture of a medicament. This medicament has the uses described herein.

More particularly, another aspect of the invention relates to the use of a compound of formula I or a pharmaceutically acceptable salt or solvate thereof, a DP antagonist and an HMG Co-A reductase inhibitor, such as simvastatin, in the manufacture of a medicament. This medicament has the uses described herein.

Compounds of the present invention have anti-hyperlipidemic activity, causing reductions in LDL-C, triglycerides, apolipoprotein a and total cholesterol, and increases in HDL-C. Consequently, the compounds of the present invention are useful in treating dyslipidemias. The present invention thus relates to the treatment, prevention or reversal of atherosclerosis and the other diseases and conditions described herein, by administering a compound of formula I or a pharmaceutically acceptable salt or solvate in an amount that is effective for treating, preventin or reversing said condition. This is achieved in humans by administering a compound of formula I or a pharmaceutically acceptable salt or solvate thereof in an amount that is effective to treat or prevent said condition, while preventing, reducing or minimizing flushing effects in terms of frequency and/or severity.

One aspect of the invention that is of interest is a method of treating atherosclerosis in a human patient in need of such treatment comprising administering to the patient a compound of formula I or a pharmaceutically acceptable salt or solvate thereof in an amount that is effective for treating atherosclerosis in the absence of substantial flushing.

Another aspect of the invention that is of interest relates to a method of raising serum HDL levels in a human patient in need of such treatment, comprising administering to the patient a compound of formula I or a pharmaceutically acceptable salt or solvate thereof in an amount that is effective for raising serum HDL levels.

Another aspect of the invention that is of interest relates to a method of treating dyslipidemia in a human patient in need of such treatment comprising administering to the patient a compound of formula I or a pharmaceutically acceptable salt or solvate thereof in an amount that is effective for treating dyslipidemia.

Another aspect of the invention that is of interest relates to a method of reducing serum VLDL or LDL levels in a human patient in need of such treatment, comprising administering to the patient a compound of formula I or a pharmaceutically acceptable salt or solvate thereof in an amount that is effective for reducing serum VLDL or LDL levels in the patient in the absence of substantial flushing.

Another aspect of the invention that is of interest relates to a method of reducing serum triglyceride levels in a human patient in need of such treatment, comprising administering to the patient a compound of formula I or a pharmaceutically acceptable salt or solvate thereof in an amount that is effective for reducing serum triglyceride levels.

Another aspect of the invention that is of interest relates to a method of reducing serum Lp(a) levels in a human patient in need of such treatment, comprising administering to the patient a compound of formula I or a pharmaceutically acceptable salt or solvate thereof in an amount that is effective for reducing serum Lp(a) levels. As used herein Lp(a) refers to lipoprotein (a).

Another aspect of the invention that is of interest relates to a method of treating diabetes, and in particular, type 2 diabetes, in a human patient in need of such treatment comprising administering to the patient a compound of formula I or a pharmaceutically acceptable salt or solvate thereof in an amount that is effective for treating diabetes.

Another aspect of the invention that is of interest relates to a method of treating metabolic syndrome in a human patient in need of such treatment comprising administering to the patient a compound of formula I or a pharmaceutically acceptable salt or solvate thereof in an amount that is effective for treating metabolic syndrome.

Another aspect of the invention that is of particular interest relates to a method of treating atherosclerosis, dyslipidemias, diabetes, metabolic syndrome or a related condition in a human patient in need of such treatment, comprising administering to the patient a compound of formula I or a pharmaceutically acceptable salt or solvate thereof and a DP receptor antagonist, said combination being administered in an amount that is effective to treat atherosclerosis, dyslipidemia, diabetes or a related condition in the absence of substantial flushing.

Another aspect of the invention that is of particular interest relates to the methods described above wherein the DP receptor antagonist is selected from the group consisting of compounds A through AJ and the pharmaceutically acceptable salts and solvates thereof.

### Methods of Synthesis for Compounds of Formula I

Compounds of formula I have been prepared by the following representative reaction schemes. It is understood that similar reagents, conditions or other synthetic approaches to these structure classes are conceivable to one skilled in the art of organic synthesis. Therefore these reaction schemes should not be construed as limiting the scope of the invention. All substituents are as defined above unless indicated otherwise.

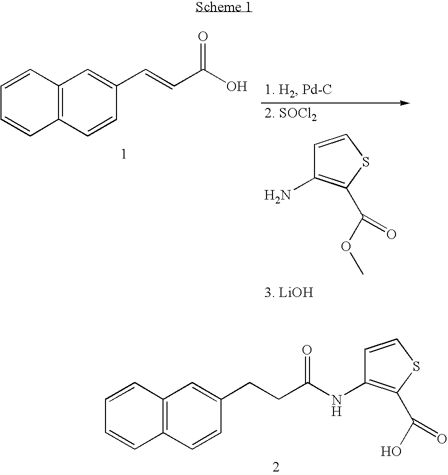

Compounds of formula I can be prepared as illustrated in Scheme 1 by reduction of an enoic acid such as 1, followed by acylation of a thiophene aminoester, and subsequent saponification to generate compounds such as the naphthyl thiophene acid 2.

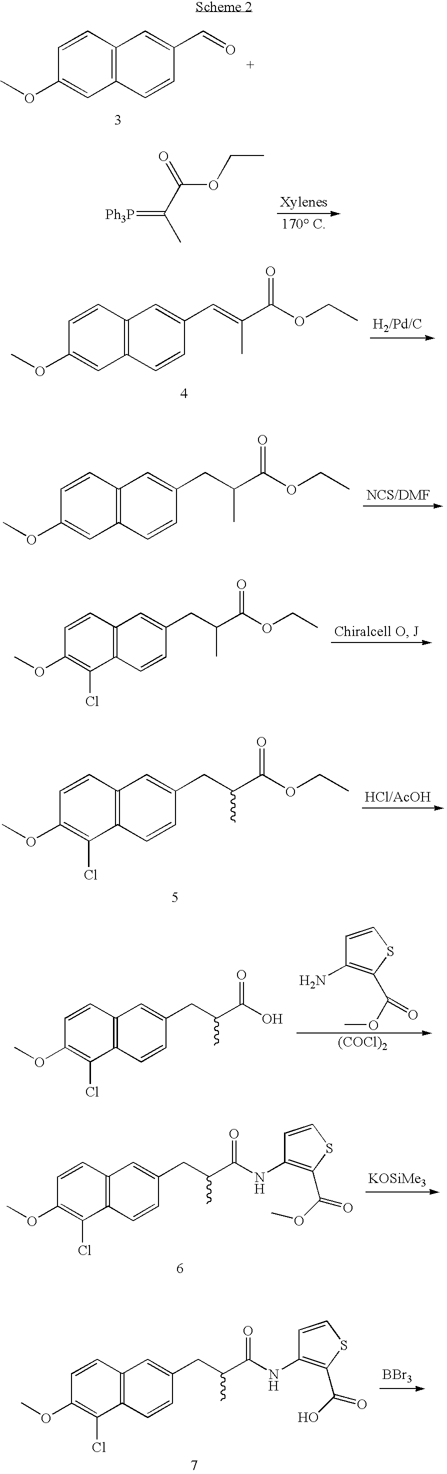

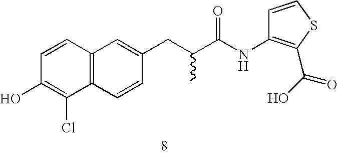

Compounds of formula I can also be prepared as illustrated in Scheme 2, beginning with homologation of an appropriate aldehyde such as 3. The resulting enoate 4 can be reduced, chlorinated, and the racemic mixture resolved to generate intermediates such as 5. Acidic hydrolysis and acylation of a thiophene aminoester may provide ester intermediates such as 6. Subsequent conversions such as demethylation of either the ester or the ether can provide carboxylic acid compounds such as 7 and 8 respectively.

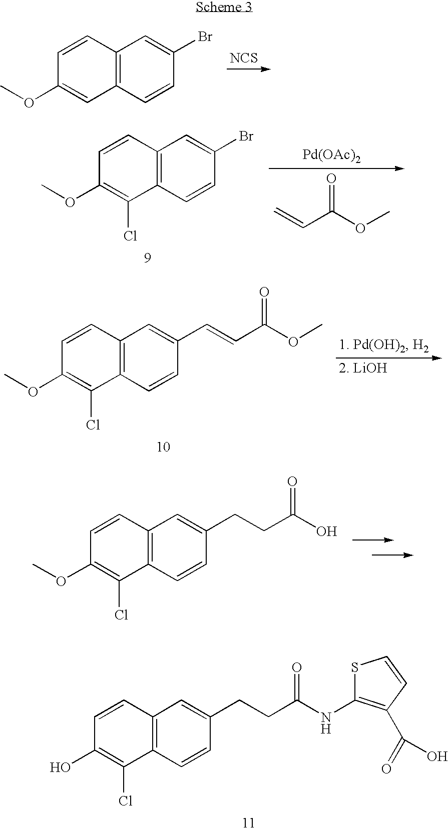

Compounds of formula I can also be prepared as illustrated in Scheme 3, through chlorination of a methoxy naphthyl precursor to obtain intermediates such as 9. This bromide 9 can undergo a palladium coupling to generate enoate intermediates such as 10. Hydrogenation, saponification and acylation of an amino thiophene can provide the desired compound such as 11, after liberation of the hydroxyl and acid functionalities.

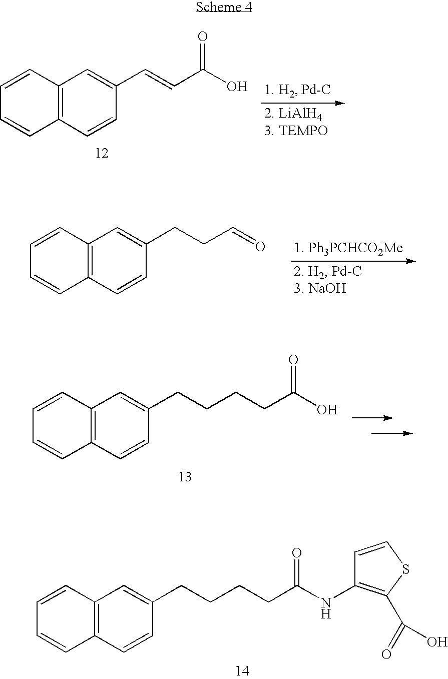

Compounds of formula I can alternatively be prepared as illustrated in Scheme 4, to access higher homolog derivatives, such as 14. An enoic acid such as 12 can be converted to its saturated aldehyde, which in turn can be homologated with a stabilized ylide, providing acid intermediates such as 13 upon hydrogenation and saponification. Acylation of an amino thiophene with 13 provides the desired compound such as 14, after saponification.

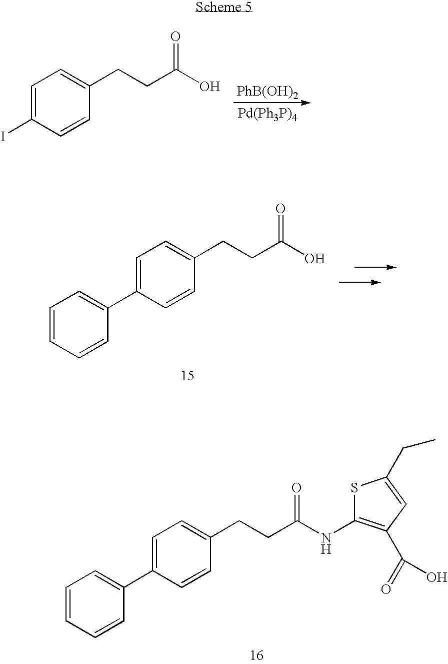

Compounds of formula I can alternatively be prepared as illustrated in Scheme 5, to access biaryl derivatives, such as 16. Intermediates such as 15 can be generated through standard palladium catalyzed aryl coupling reactions with appropriate boronic acids.

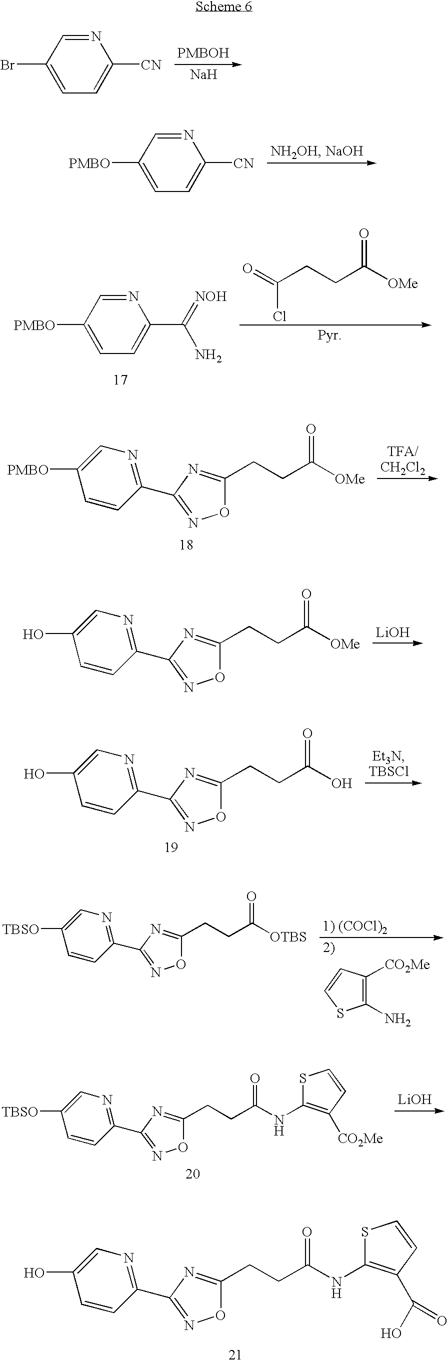

Compounds of formula I can also be prepared as illustrated in Scheme 6 to access heterocyclic biaryl derivatives. A pyridyl intermediate, such as hydroxy amidine 17, can be generated en route toward oxadiazole 18. Deprotection to give 19, allows the acylation of a thiophene amino ester to provide 20, followed by saponification toward products such as 21.

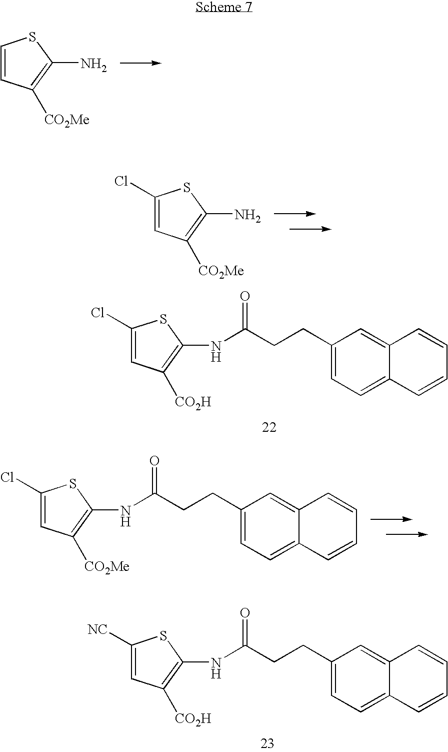

Compounds of formula I can alternatively be prepared as illustrated in Scheme 7, to access substituted thiophene derivatives. Chlorination of a thiophene amino ester can lead to derivatives such as 22. Cyanation of such chloride intermediates allows the generation of nitrile derivatives exemplified by 23.

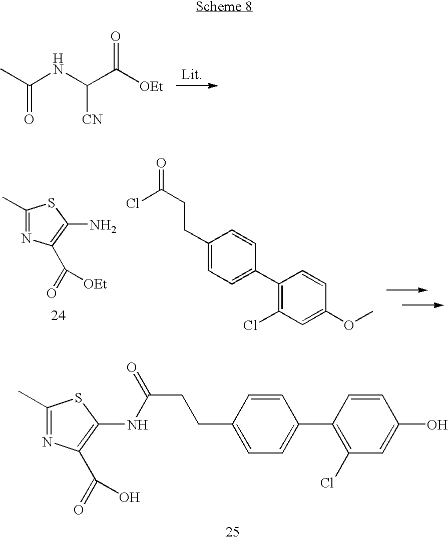

Compounds of formula I can additionally be prepared as illustrated in Scheme 8, to access thiazole derivatives. Literature methods to synthesize 24 allow access to compounds such as 25, following typical acylation, ether demethylation and saponification reactions known to those skilled in the art.

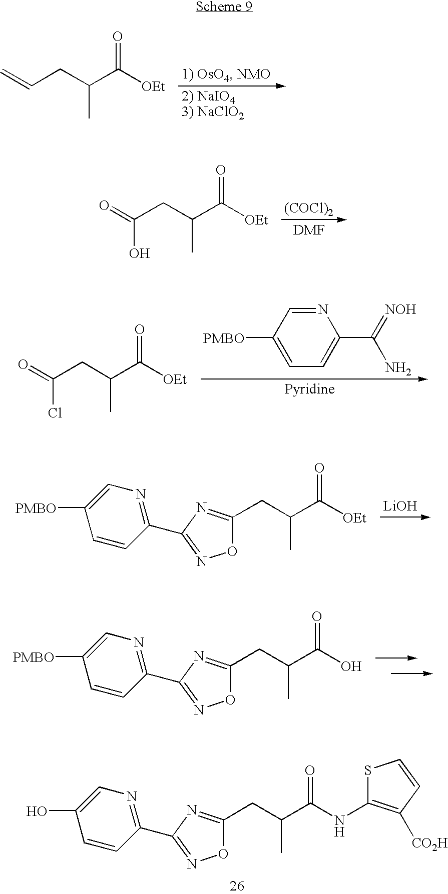

Compounds of formula I can also be prepared as illustrated in Scheme 9, to access chain-substituted biheteroaryl derivatives such as 26.

The various organic group transformations and protecting groups utilized herein can be performed by a number of procedures other than those described above. References for other synthetic procedures that can be utilized for the preparation of intermediates or compounds disclosed herein can be found in, for example, M. B. Smith, J. March Advanced Organic Chemistry, 5^{th} Edition, Wiley-Interscience (2001); R. C. Larock Comprehensive Organic Transformations, A Guide to Functional Group Preparations, 2^{nd} Edition, VCH Publishers, Inc. (1999); T. L. Gilchrist Heterocyclic Chemistry, 3^{rd} Edition, Addison Wesley Longman Ltd. (1997); J. A. Joule, K. Mills, G. F. Smith Heterocyclic Chemistry, 3^{rd} Edition, Stanley Thornes Ltd. (1998); G. R. Newkome, W. W. Paudler Contemporary Heterocyclic Chemistry, John Wiley and Sons (1982); or Wuts, P. G. M.; Greene, T. W.; Protective Groups in Organic Synthesis, 3^{rd} Edition, John Wiley and Sons, (1999), all six incorporated herein by reference in their entirety.

### REPRESENTATIVE EXAMPLES

The following examples are provided to more fully illustrate the present invention, and shall not be construed as limiting the scope in any manner. Unless stated otherwise:

(i) all operations were carried out at room or ambient temperature, that is, at a temperature in the range 18-25° C.;

(ii) evaporation of solvent was carried out using a rotary evaporator under reduced pressure (4.5-30 mmHg) with a bath temperature of up to 50° C.;

(iii) the course of reactions was followed by thin layer chromatography (TLC) and/or tandem high performance liquid chromatography (HPLC) followed by mass spectroscopy (MS), herein termed LCMS, and any reaction times are given for illustration only;

(iv) yields, if given, are for illustration only;

(v) the structure of all final compounds was assured by at least one of the following techniques: MS or proton nuclear magnetic resonance (¹H NMR) spectrometry, and the purity was assured by at least one of the following techniques: TLC or HPLC;

(vi) ¹H NMR spectra were recorded on either a Varian Unity or a Varian Inova instrument at 500 or 600 MHz using the indicated solvent; when line-listed, NMR data is in the form of delta values for major diagnostic protons, given in parts per million (ppm) relative to residual solvent peaks (multiplicity and number of hydrogens); conventional abbreviations used for signal shape are: s. singlet; d. doublet (apparent); t. triplet (apparent); m. multiplet; br. broad; etc.;

(vii) MS data were recorded on a Waters Micromass unit, interfaced with a Hewlett-Packard (Agilent 1100) HPLC instrument, and operating on MassLynx/OpenLynx software; electrospray ionization was used with positive (ES+) or negative ion (ES−) detection; the method for LCMS ES+ was 1-2 mL/min, 10-95% B linear gradient over 5.5 min (B=0.05% TFA-acetonitrile, A=0.05% TFA-water), and the method for LCMS ES− was 1-2 mL/min, 10-95% B linear gradient over 5.5 min (B=0.1% formic acid-acetonitrile, A=0.1% formic acid-water), Waters XTerra C18-3.5 um-50×3.0 mmID and diode array detection;

(viii) the purification of compounds by preparative reverse phase HPLC(RPHPLC) was conducted on either a Waters Symmetry Prep C18-5 um-30×100 mmID, or a Waters Atlantis Prep dC18-5 um-20×100 mmID; 20 mL/min, 10-100% B linear gradient over 15 min (B=0.05% TFA-acetonitrile, A=0.05% TFA-water), and diode array detection;

(ix) automated purification of compounds by preparative reverse phase HPLC was performed on a Gilson system using a YMC-Pack Pro C18 column (150×20 mm i.d.) eluting at 20 mL/min with 0-50% acetonitrile in water (0.1% TFA);

(x) the purification of compounds by preparative thin layer chromatography (PTLC) was conducted on 20×20 cm glass prep plates coated with silica gel, commercially available from Analtech;

(xi) column chromatography was carried out on a Biotage cartridge system;

(xii) chemical symbols have their usual meanings; the following abbreviations have also been used v (volume), w (weight), b.p. (boiling point), m.p. (melting point), L (litre(s)), mL (millilitres), g (gram(s)), mg (milligrams(s)), mol (moles), mmol (millimoles), eq or equiv (equivalent(s)), IC50 (molar concentration which results in 50% of maximum possible inhibition), EC50 (molar concentration which results in 50% of maximum possible efficacy), uM (micromolar), nM (nanomolar);

(xiii) definitions of acronyms are as follows:

TEMPO is 2,2,6,6-tetramethyl-1-piperidinyloxy, free radical;

### NCS is N-chlorosuccinimide;

### NMO is N-methylmorpholine N-oxide;

TBS is tert-butyldimethylsilyl;
THF is tetrahydrofuran;
DMF is dimethylformamide;
TFA is trifluoroacetic acid;
DMSO is dimethyl sulfoxide.
PMBOH is p-methoxybenzyl alcohol and PMBO is para methoxybenzyloxy.

### Example 1

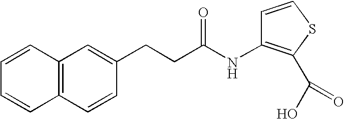

To a solution of 3-(2-naphthyl)acrylic acid (1.5 g, 7.56 mmol) in 1:1 ethanol-ethyl acetate (50 mL) was added Pd/C and the resulting mixture stirred under a H₂ balloon for 18 hours. The reaction mixture was filtered through celite, and concentrated in vacuo to give the desired propionic acid as a white solid. A solution of this acid (197 mg, 1.0 mmol) and thionyl chloride (0.7 mL) in toluene (5 mL) was heated at reflux for 4 h, cooled, concentrated in vacuo, and the excess thionyl chloride removed by azeotrope with toluene (3×3 mL). The yellow oil was diluted into toluene (3 mL), and combined with the requisite thiophene amino ester (52 mg, 0.33 mmol) as shown in Scheme 1. The reaction mixture was heated (microwave, 300 W) for 10 min at 150° C., cooled, concentrated in vacuo, and the residue purified by preparative RPHPLC. The methyl ester (34 mg, 0.1 mmol) was saponified at room temperature using excess 1N aqueous lithium hydroxide in (3:1:1) THF-methanol-water. The reaction mixture was concentrated in vacuo to remove volatiles, acidified with 1N aqueous HCl to pH=7, and purified by preparative RPHPLC. ¹H NMR (acetone-d₆, 500 MHz) δ 10.3 (1H, s), 8.14 (1H, d), 7.83 (4H, m) 7.75 (1H, d), 7.46 (3H, m), 3.22 (2H, t), 2.91 (2H, t); LCMS m/z 326 (M+1).

### Examples 2-6

The following compounds were prepared under conditions similar to those described in Example 1 above, and illustrated in Scheme 1.

| | | LCMS |
|---|---|---|
| EXAMPLE | | (m/z) |
| 2 | 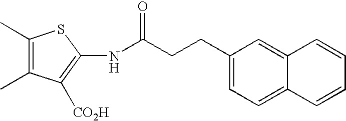 | 352 (M − 1) |
| 3 | 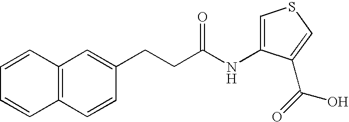 | 326 (M + 1) |
| 4 | 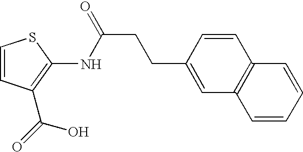 | 326 (M + 1) |
| 5 | 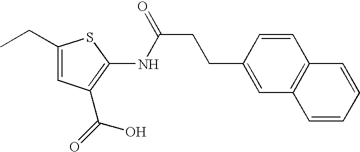 | 352 (M − 1) |
| 6 | 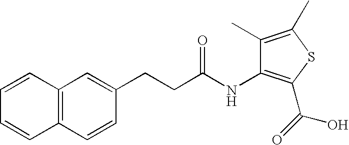 | 352 (M − 1) |

NMR data for selected Examples:

### Example 2

¹H NMR (DMSO-d₆, 500 Mz) δ 7.85-7.80 (m, 3H), 7-74 (s, 1H), 7.48-7.41 (m, 3H), 3.08 (t, 2H), 2.91 (t, 2H), 2.19 (s, 3H), 2.16 (s, 3H).

### Example 3

¹H NMR (acetone-d₆, 500 MHz) δ 10.2 (1H, s), 8.27 (1H, d), 8.04 (1H, d), 7.82 (4H, m), 7.45 (3H, m), 3.21 (2H, t), 2.87 (2H, t).

### Example 4

¹H NMR (acetone-d₆, 500 MHz) δ 11.1 (1H, s), 7.85 (4H, m), 7.48 (3H, m), 7.22 (1H, d), 6.91 (1H, d), 3.25 (2H, t), 3.03 (2H, t).

### Example 5

¹H NMR (500 MHz, CD₃OD) δ 7.86-7.83 (m, 3H), 7.77 (s, 1H), 7.51-7.45 (m, 3H), 6.90 (s, 1H), 3.24 (t, 2H), 2.97 (t, 2H), 2.77 (q, 2H), 1.31 (t, 3H).

### Example 6

¹H NMR (DMSO-d₆, 500 Mz) δ 7.83-7.79 (m, 3H), 7.73 (s, 1H), 7.47-7.41 (m, 3), 3.20 (t, 2H), 2.91 (t, 2H), 2.25 (s, 3H), 2.25 (s, 3H).

### Example 7

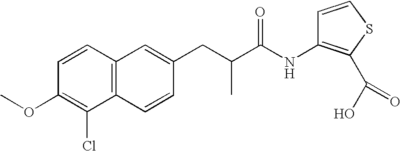

To a xylenes solution of 6-methoxy-2-naphthaldehyde (0.855 g, 4.585 mmol) was added the stabilized ylide shown in Scheme 2 (2.16 g, 5.96 mmol, 1.3 eq.) at room temperature. The solution was heated to reflux for 4 h. The solvent was removed under vacuum, and the residue was chromatographed with AcOEt/Hexanes (4 to 1) to obtain the ethyl enoate intermediate. To a methanol solution of this intermediate (5.73 g) was added Pd/C (0.3 g), and the mixture was subjected to hydrogenation under a balloon atmosphere of H₂ gas, at room temperature for 16 h. The solution was filtered, and the solvent was removed in vacuo to obtain the saturated ester. The methoxy naphthyl ester (5.73 g) was treated with NCS (0.82 g, 6.11 mmol, 1.1 eq) in DMF solvent at room temperature, and the solution was stirred for 16 h. Removal of the DMF in vacuo provided a residue which was recrystallized from methanol/methylene chloride to obtain the chlorinated intermediate. The racemic mixture of this chloride (1.5 g, 3.69 mmol) was separated into its single enantiomers using chiral HPLC with a Chiralcel OJ column, and isocratic elution with 35% isopropanol-heptane. The ethyl ester intermediate (65 mg, 0.21 mmol) was dissolved in (1:1) acetic acid-HCl (2 mL) and heated to 110° C. for 10 min. Then 5 mL of water was added, and the solution cooled to 0° C. to obtain the acid intermediate after filtration. Oxalyl chloride (0.3 mmol) was then added to a CH₂Cl₂ (2 mL) solution of this acid intermediate (45 mg, 0.1 mmol), and one drop of DMF was added at 0° C. The solvent was removed in vacuo after the solution was stirred for 1 h at room temperature. The residue was dissolved in THF (2 mL), and this solution was added to a THF (2 mL) solution of 3-amino-2-carboxylthiophene (0.11 mmol) and Et₃N (0.3 mmol) at 0° C. The pure thiophene methyl ester intermediate was obtained after HPLC purification. Potassium trimethylsilanolate (4 eq, 0.4 mmol) was added to a THF solution of this methyl ester intermediate (0.09 mmol) at 0° C. The solution was stirred at room temperature for 2 h, and the desired product was obtained by preparative RPHPLC purification. ¹H NMR (CD₃OD, 500 MHz) δ 8.07 (d, 1H), 7.99 (d, 1H), 7.74 (d, 1H), 7.66 (s, 1H), 7.64 (d, 1H), 7.47 (dd 1H), 7.40 (d, 1H), 4.00 (s, 3H), 3.17 (m, 1H), 2.97 (m, 2H), 1.30 (d, 3H); LCMS m/z 402 (M−1).

### Example 8

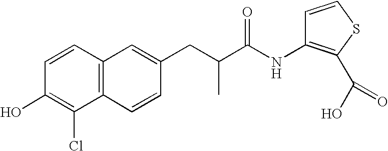

Boron tribromide (1M CH₂Cl₂ solution, 0.3 mL) was added to a CH₂Cl₂ solution of EXAMPLE 7 (0.05 mmol) at 0° C., and the solution was stirred for 3 h. The reaction mixture was slowly warmed to room temperature for 20 min, and cooled to 0° C. again. Then five drops of methanol was added, followed by adding 5 mL of water. The pure desired product was obtained by preparative RPHPLC purification. ¹H NMR (CD₃OD, 500 MHz) δ 10.38 (s, 1H), 7.98 (d, 1H), 7.97 (d, 1H), 7.60 (d, 1H), 7.58 (s, 1H), 7.56 (d, 1H), 7.39 (d, 1H), 7.13 (d, 1H), 3.13 (m, 1H), 2.90 (m, 2H), 1.27 (d, 3H); LCMS m/z 388 (M−1).

### Examples 9-10

The following compounds were prepared under conditions similar to those described in Examples 7-8 above, and illustrated in Scheme 2.

| EXAMPLE | | LCMS (m/z) |
|---|---|---|
| 9 | 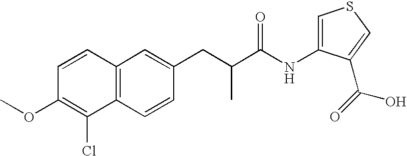 | 402 (M − 1) |
| 10 | 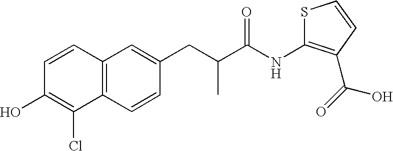 | 388 (M − 1) |

NMR data for selected Examples:

### Example 9

¹H NMR (CD₃OD, 500 MHz) δ 8.07 (d, 1H), 7.74 (d, 1H), 7.65 (s, 1H), 7.46 (dd, 1H), 7.40 (d, 1H), 7.16 (d, 1H), 6.84 (d, 1H), 4.00 (s, 3H), 3.18 (m, 1H), 3.02 (m, 2H), 1.25 (d, 3H).

### Example 10

¹H NMR (CD₃OD, 500 MHz) δ 11.12 (s, 1H), 7.98 (d, 1H), 7.56 (s, 1H), 7.49 (d, 1H), 7.39 (dd, 1H), 7.14 (t, 2H), 6.82 (d, 1H), 3.14 (m, 1H), 2.97 (m, 2H), 1.29 (d, 3H).

### Example 11

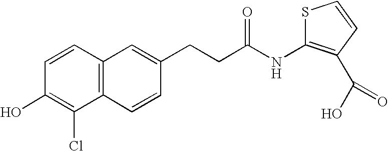

The chlorination of 2-bromo-6-methoxynaphthalene with NCS followed a similar procedure that was described in EXAMPLE 7 and also has been described in the literature: Vyas, P. V.; Bhatt, A. K.; Ramachandraiah, G.; Bedekar, A. V. Tetrahedron Letters 2003, 44(21), 4085-4088. As shown in Scheme 3, this bromide intermediate (6 g, 22.1 mmol) was combined with methyl acrylate (5.9 mL, 66.4 mmol), triethylamine (60 mL), tris(O-tolyl)phosphorus(U) ligand (120 mg), and palladium(II) acetate (226 mg). The reaction mixture was heated at 100° C. for 15 h in a sealed tube under an argon atmosphere, then cooled, partitioned between water and ethyl acetate, the precipitate collected, and purified by column chromatography (SiO₂, ethyl acetate-hexane). As in the Examples above, this enoate intermediate was hydrogenated under a balloon of H₂ gas, followed by saponification with lithium hydroxide, and the saturated acid converted to the thiophene amide product under oxalyl chloride mediated activation. The product was obtained after BBr₃-mediated demethylation of the ether as in the Examples above, and the product purified by preparative RPHPLC. ¹H NMR (CD₃OD, 600 MHz) δ 8.00 (d, 1H), 7.63 (d, 11H), 7.60 (d, 1H), 7.45 (dd, 11H), 7.16 (d, 11H), 7.13 (d, 1H), 6.82 (d, 1H), 3.16 (t, 2H), 2.90 (t, 2H); LCMS m/z 376 (M+1).

### Example 12

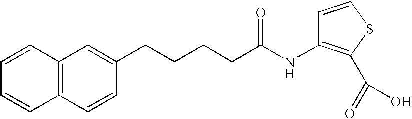

Commercially available 3(2-naphthyl)acrylic acid (5 g) in 50 mL of (1:1) methanol-methylene chloride was treated with catalytic palladium on carbon, and hydrogenated at 1 atmosphere with a hydrogen-filled balloon for 12 h. The reaction mixture was filtered over celite and concentrated in vacuo to provide the clean crude acid. This intermediate (1 g, 5 mmol) in diethyl ether (100 mL) was added dropwise to a solution of lithium aluminum hydride (380 mg, 10 mmol) in 100 mL of anhydrous diethyl ether under nitrogen atmosphere. The reaction mixture was aged for 12 h, quenched with aqueous Rochelle salt, stirred for an additional 2 h, partitioned between saturated aqueous NaHCO₃ and diethyl ether, the organic phase was separated and dried over anhydrous sodium sulfate, and then evaporated under reduced pressure to provide the crude alcohol product. This alcohol (1.0 g, 5.4 mmol) was oxidized directly with iodobenzene diacetate (1.7 g, 5.9 mmol) and catalytic TEMPO (10%) in methylene chloride solvent (30 mL). After 2 h, the reaction mixture was quenched with aqueous sodium thiosulfate, partitioned with methylene chloride, the organic phase washed with aqueous NaHCO₃, and the organic phase concentrated in vacuo to provide the clean aldehyde product as an oil. This crude aldehyde intermediate (240 mg, 1.3 mmol) was combined with methyl (triphenylphosphoranylidene) acetate (650 mg, 1.94 mmol) in toluene (5 mL), and the reaction mixture heated at reflux for 2 h. The mixture was concentrated in vacuo to a residue which was purified by flash column chromatography (SiO₂, EtOAc/hexanes) to give the desired methyl enoate. This intermediate was then treated with catalytic palladium on carbon in methanol (10 mL), and hydrogenated at 1 atmosphere with a hydrogen-filled balloon for 4 h. The reaction mixture was filtered over celite and concentrated in vacuo to provide the clean crude ester which was dissolved in (3:1:1) TIF-MeOH—H₂O (10 mL), treated with aqueous 1N NaOH (2.6 mL), aged for 6 h, the mixture acidified and extracted with diethyl ether. The organic phase was concentrated in vacuo to provide the clean acid, which is defined as Compound 13 in Scheme 4. This intermediate acid was converted into EXAMPLE 12 in a manner similar to the Examples above. The compound was purified via preparative RPHFLC to give the desired product. ¹H NMR (CD₃OD, 500 MHz) δ 10.42 (s, 1H), 7.99 (d, 1H), 7.78 (d, 1H), 7.76 (d, 2H), 7.65 (d, 1H), 7.64 (s, 1H), 7.41 (m, 2H), 7.35 (dd, 1H), 2.84 (t, 2H), 2.51 (t, 2H), 1.80 (m, 4H); LCMS m/z 354 (M+1).

### Examples 13-15

The following compounds were prepared under conditions similar to those described in the Examples above, and illustrated in Scheme 4.

| EXAMPLE | | LCMS (m/z) |
|---|---|---|
| 13 | 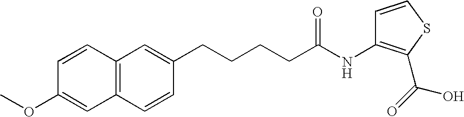 | 382 (M − 1) |
| 14 | 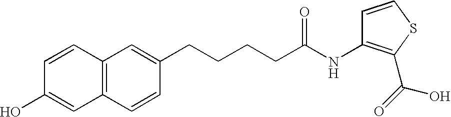 | 368 (M − 1) |
| 15 | 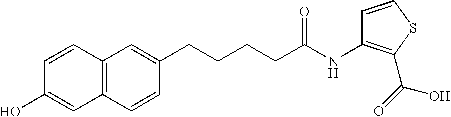 | 368 (M − 1) |

NMR data for selected Examples:

### Example 13

¹H NMR (CD₃OD, 500 MHz) δ 8.02 (d, 1H), 7.66 (m, 3H), 7.57 (s, 1H), 7.31 (dd, 1H), 7.18 (s, 1H), 7.08 (dd, 1H), 3.90 (s, 3H), 2.80 (t, 2H), 2.51 (t, 2H), 1.80 (m, 4H).

### Example 14

¹H NMR (CD₃OD, 500 MHz) δ 8.00 (d, 1H), 7.63 (m, 2H), 7.55 (d, 1H), 7.52 (s, 1H), 7.24 (dd, 1H), 7.06 (d, 1H), 7.02 (dd, 1H), 2.77 (t, 2H), 2.49 (t, 2H), 1.78 (m, 4H).

### Example 15

¹H NMR (CD₃OD, 500 MHz) δ 7.62 (d, 1H), 7.55 (d, 1H), 7.52 (s, 1H), 7.25 (dd, 1H), 7.21 (d, 1H), 7.05 (m, 1H), 7.01 (dd, 1H), 6.84 d (d, 1H), 2.77 (t, 2H), 2.57 (t, 2H), 1.79 (m, 4H).

### Example 16

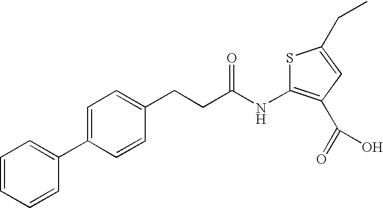

Commercially available 3-(4-iodophenyl)propionic acid (200 mg, 0.72 mmol) was combined with phenyl boronic acid (177 mg, 1.45 mmol), catalytic tetrakis-(triphenylphosphine)-palladium (20 mg), and saturated aqueous sodium bicarbonate (1M, 1.45 mL, 1.45 mmol) in (1:1) dioxane-ethanol (5 mL). The reaction mixture was heated at 100° C. overnight, cooled to room temperature, filtered, and concentrated in vacuo. The residue was purified via preparative RPHPLC to give the biaryl propionic acid intermediate, which is defined as Compound 15 in Scheme 5. This intermediate acid was converted into EXAMPLE 16 in a manner similar to the Examples above. The compound was purified via preparative RPHPLC to give the desired product. ¹H NMR (CD₃OD, 500 MHz) δ 7.68-7.62 (m, 4H), 7.51-7.38 (m, 5H), 6.93 (s, 1H), 3.12 (t, 2H), 2.93 (t, 2H), 2.80 (q, 2H), 2.66 (s, 1H), 1.33 (t, 3H); LCMS m/z 378 (M−1).

### Examples 17-20

The following compounds were prepared under conditions similar to those described in the Examples above, and illustrated in Scheme 5.

| | | LCMS |
|---|---|---|
| EXAMPLE | | (m/z) |
| 17 | 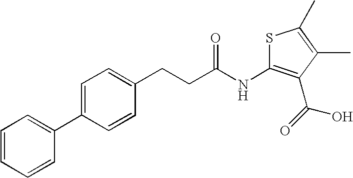 | 378 (M − 1) |
| 18 | 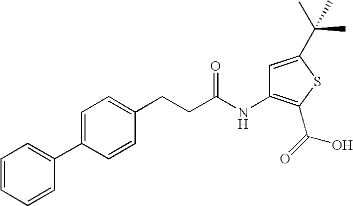 | 406 (M − 1) |
| 19 | 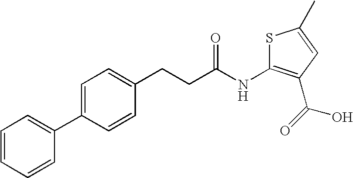 | 365 (M − 1) |
| 20 | 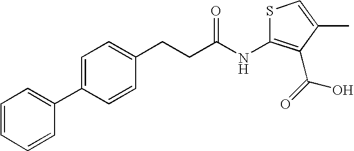 | 364 (M − 1) |

NMR data for selected Examples:

### Example 17

¹H NMR (DMSO-d₆, 500 MHz) δ 7.59 (d, 1H), 7.55 (d, 1H), 7.42 (t, 2H), 7.35-7.30 (m, 3H), 3.07 (t, 2H), 2.84 (t, 2H), 2.62 (m, 1H), 2.56 (s, 6H).

### Example 18

¹H NMR (DMSO-d₆, 500 MHz) δ 7.80 (s, 1H), 7.61 (d, 2H), 7.56 (d, 2H), 7.43 (t, 2H), 7.35-7.31 (m, 3H), 2.95 (t, 2H), 2.78 (t, 2H), 1.32 (s, 9H).

### Example 19

¹H NMR (500 Mz, CD₃OD) δ 7.60-7.54 (m, 4H), 7.44-7.31 (m, 5H), 6.87 (s, 1H), 3.09 (t, 2H), 2.86 (t, 2H), 2.39 (s, 3H).

### Example 20

¹H NMR (DMSO-d₆, 500 Mz) δ 7.56 (d, 1H), 7.52 (d, 1H), 7.40 (t, 2H), 7.33-7.28 (m, 3H), 6.49 (s, 1H), 3.07 (t, 2H), 2.84 (t, 2H), 2.35 (s, 3H).

### Example 21

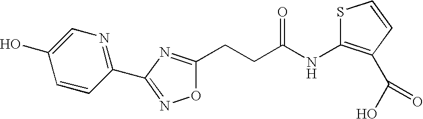

To NaH (7.2 g, 60%) was added DMF (100 mL) followed by 4-methoxybenzyl alcohol (18.7 mL) at 0° C. After 25 min at 0° C., the mixture was warmed to rt and stirred for additional 30 min. To the resulting solution was added 5-bromo-2-cyanopyridine (22.9 g) in one portion. The reaction was exothermic and stirred for 10 min before it was cooled to rt. The mixture was diluted with 500 mL of ethyl acetate, washed with water (500 mL×3). The first two aqueous phases were extracted with dichloromethane (500 mL×2). The combined dichloromethane phase was washed with water (500 mL×3). The combined organic phases were dried over sodium sulfate and concentrated to give 4-(4-methoxybenzyloxy)-2-cyanopyridine (22.6 g) as a white solid. To the suspension of 4-(4-methoxybenzyloxy)-2-cyanopyridine (24.6 g) and hydroxylamine hydrochloride (8.55 g) in ethanol (500 mL) was added NaOH (4.92 g in 50 mL of water) dropwise. The mixture was stirred at rt overnight. The solid was collected by filtration to give 4-(4-methoxybenzyloxy)-2-hydroxyamidinylpyridine 17 as a white solid.

To a solution of this intermediate (180 mg, 0.66 mmol) in 8 mL of pyridine was added the mono acyl chloride (199 mg, 1.32 mmol). The resulting mixture was heated at 130° C. for 30 min. After removing most solvent, the residue was diluted with dichloromethane and purified by Biotage chromatography (10-50% ethyl acetate in hexane) to afford the oxadiazole intermediate as a white solid. To this oxadiazole intermediate (126 mg, 0.34 mmol) was added 4 mL of a mixture of trifluoroacetic acid and dichloromethane (1:1) at 23° C. After 30 min, the purple colored reaction mixture was concentrated in vacuo. The residue was used directly in the next step without further purification. To a mixture of this crude hydroxypyridine methyl ester in 20 mL of THF:methanol:water (3:1:1), was added a solution of lithium hydroxide (5 mL, 1N). After 1 h, most of the volatiles were removed in vacuo. To the residue was added 15 mL of water, and the mixture was extracted with 30% isopropanol in chloroform (3×50 mL). The combined organic phase was concentrated, and the residue was purified by RPHPLC to give the acid intermediate as a colorless oil. To a mixture of this acid (68 mg, 0.29 mmol) in 10 mL of dichloromethane, were added triethylamine (102 mg, 0.14 mL) and tert-butyldimethylsilyl chloride (109 mg, 0.73 mmol) at 23° C. After 3 h the mixture was quenched with water, and the aqueous layer was extracted with dichloromethane. The combined organic phase was concentrated in vacuo to give the bis-TBS-protected product as a brown oil, which was directly used in the next step. In an ice bath, to this intermediate in dichloromethane (5 mL), was added one drop of DMF, and then a solution of oxalyl chloride (0.28 mL, 2 N in dichloromethane). After 1.5 h, the mixture was warmed to 23° C. and stirred for another 1.5 h. The resulting mixture was concentrated in vacuo, and then dissolved in dichloromethane (5 mL). To the resulting solution was then added methyl-2-aminothiophene carboxylate (88 mg, 0.56 mmol). The reaction mixture was stirred overnight, and the solvent was then removed, and the crude residue was dissolved in 10 mL of THF:methanol:water (3:1:1). To this solution was added aqueous lithium hydroxide (6 mL, 1N). After 1 h, most of the volatiles were removed in vacuo. To the residue was added 5 mL of water, and the mixture was extracted with 30% isopropanol in chloroform (3×10 mL). The combined organic phase was concentrated, and the residue was purified by RPHPLC to give the desired compound as a light brown solid. ¹H NMR (acetone-d₆, 500 MHz) δ 11.2 (1H, s), 8.35 (1H, s), 7.99 (1H, d), 7.40 (1H, dd), 7.21 (1H, d), 6.91 (1H, d), 3.42 (2H, t), 3.29 (2H, t); LCMS m/Z 361 (M+1).

### Examples 22-23

The following compounds were prepared under conditions similar to those described in the Examples above, and illustrated in Scheme 6.

| | | LCMS |
|---|---|---|
| EXAMPLE | | (m/z) |
| 22 | 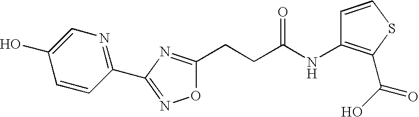 | 361 (M + 1) |
| 23 | 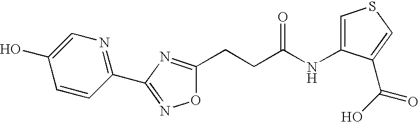 | 361 (M + 1) |

NMR data for selected Examples:

### Example 22

¹H NMR (DMSO-d₆, 500 MHz) δ 10.6 (1H, bs), 10.3 (1H, s), 8.24 (1H, d), 7.88 (2H, m), 7.81 (1H, d), 7.28 (1H, dd), 3.26 (2H, t), 3.07 (2H, t).

### Example 23

¹H NMR (DMSO-d₆, 500 MHz) δ 10.6 (1H, bs), 10.2 (1H, s), 8.27 (2H, m), 7.85 (2H, m), 7.29 (1H, dd), 3.26 (2H, t), 3.02 (2H, t).

### Example 24

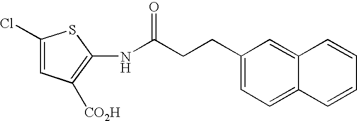

As shown in Scheme 7, N-chlorosuccinimide (105 mg, 0.793 mmol) was added to the commercially available thiophene aminoester (204 mg, 0.793 mmol) in tetrahydrofuran (8 mL). The reaction mixture was stirred overnight, and the solvent was removed. The chloride intermediate was purified by silica gel chromatography. This chloro aminoester was acylated and saponified under conditions described in the Examples above to provide EXAMPLE 24. ¹H NMR (CD₃OD, 500 MHz) δ 7.86-7.81 (m, 3H), 7.75 (s, 1H), 7.49-7.44 (m, 3H), 7.11 (s, 1H), 3.10 (t, 2H), 3.02 (t, 2H); LCMS m/z 358 (M−1).

### Example 25

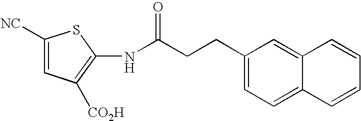

As shown in Scheme 7, the chloro amidoester (120 mg, 0.321 mmol) was combined with zinc cyanide (30 mg, 0.257 mmol), 1,1′-Bi(diphenylphosphino)ferrocene (71 mg, 0.128), in N,N′-dimethylacetamide (3 mL), and the solution degassed. The catalyst, tris(dibenzylideneacetone)-dipalladium (59 mg, 0.064 mmol) was added, and the solution again degassed. The reaction mixture was heated in a microwave reactor at 60 W and 170° C. for 1 h. The reaction mixture was quenched with a (1:5) NH₄OH:water solution, diluted with ethyl acetate, and the ethyl acetate layer washed with brine. The organic layer was dried over Na₂SO₄, concentrated, and the product purified by silica gel chromatography. This cyano amidoester was saponified under conditions described in the Examples above to provide EXAMPLE 25. ¹H NMR (CD₃OD, 500 MHz) δ 7.84 (s, 1H) 7.80-7.76 (m, 3H), 7.71 (s, 1H), 7.45-7.39 (m, 3H), 3.22 (t, 2H), 3.01 (t, 2M); LCMS m/z 350 (M−1).

### Example 26

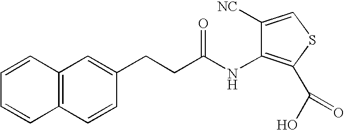

EXAMPLE 26 was prepared under conditions similar to those described in the Examples above, and illustrated in Scheme 7. ¹H NMR (DMSO-d₆, 500 MHz) δ 8.28 (s, 1H), 7.86-7.82 (m, 3H), 7.70 (s, 1H), 7.48-7.42 (m, 3H), 3.12 (t, 2H), 2.90 (t, 2H); LCMS m/z 350 (M−1).

### Example 27

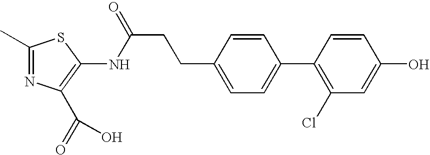

Shown in Scheme 8, the acetamido cyanoester was converted to the requisite thiazole aminoester following literature procedures: Golankiewicz, Bozenna; Januszczyk, Piotr; Gdaniec, Maria; Kosturkiewicz, Zofia Tetrahedron EN; 41(24), 1985, 5989. This thiazole aminoester intermediate was coupled under similar conditions described in the Examples above, with the methoxychlorobiphenyl acid chloride shown in Scheme 8, itself prepared under similar Suzuki conditions also described in the Examples above. The resultant amidobiaryl methyl ether was demethylated with BBr₃ under similar conditions described above, and the product was saponified and purified via preparative RPHPLC. ¹H NMR (CD₃OD, 500 MHz) δ 7.29 (s, 4H), 7.22 (d, 1H), 7.02 (d, 1H) 6.91 (dd, 1H), 3.07 (t, 2H), 2.90 (t, 2H), 2.59 (s, 3H); LCMS m/z 415 (M−1).

### Examples 28-34

The following compounds were prepared under conditions similar to those described in the Examples above.

| EXAMPLE | | LCMS (m/z) |
|---|---|---|
| 28 | 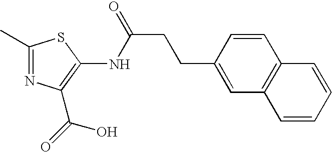 | 339 (M − 1) |
| 29 | 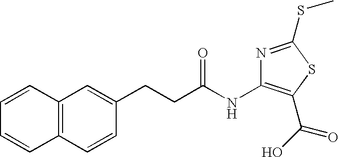 | 373 (M + 1) |
| 30 | 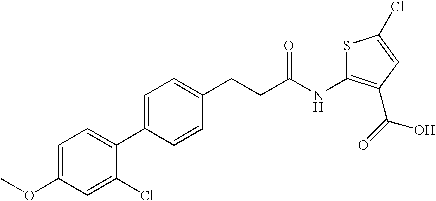 | 448 (M − 1) |
| 31 | 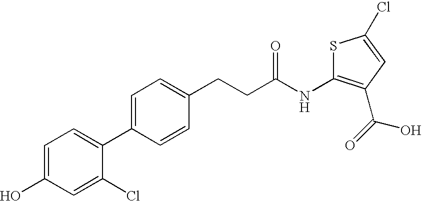 | 433.9 (M − 1) |
| 32 | 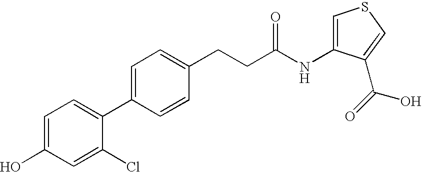 | 402 (M + 1) |
| 33 | 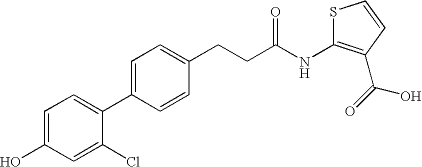 | 402 (M + 1) |
| 34 | 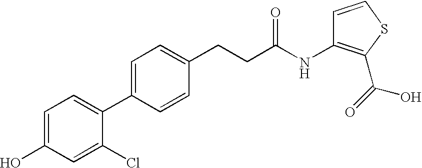 | 402 (M + 1) |

NMR data for selected Examples:

### Example 28

¹H NMR (500 MHz, CD₃OD) δ 7.81-7.76 (m, 3H), 7.71 (s, 1H), 7.45-7.37 (m, 3H), 3.21 (t, 2H), 2.98 (t, 2H), 2.59 (s, 3H).

### Example 29

¹H NMR (DMSO-d₆, 500 Mz) δ 7.87-7.83 (m, 12H), 7.74 (s, 1H), 7.48-7.43 (3H), 3.06 (t, 2H), 2.89 (t, 2H), 2.68 (s, 3H).

### Example 30

¹H NMR (500 MHz, CD₃OD) δ 7.31 (s, 4H), 7.25 (d, 1H), 7.07 (s, 1H), 7.05 (d, 1H) 6.93 (dd, 1H), 3.83 (s, 3H), 3.08 (t, 21), 2.89 (t, 2H), 2.63 (m, 1H).

### Example 31

¹H NMR (500 MHz, CD₃OD) δ 7.28 (s, 4H), 7.12 (d, 1H), 7.05 (s, 1H), 6.88 (d, 1H) 6.75 (dd, 1H), 3.06 (t, 2H), 2.87 (t, 2H).

### Example 32

¹H NMR (CD₃OD, 500 MHz) δ 8.18 (1H, d), 7.92 (1H, dd), 7.28 (4H, s), 7.12 (1H, d), 6.88 (1H, d), 6.76 (1H, dd), 3.06 (2H, t), 2.76 (2H, t).

### Example 33

¹H NMR (CD₃OD, 500 MHz) δ 7.28 (4H, s), 7.24 (1H, d), 7.16 (1H, d), 6.88 (1H, d), 6.84 (1H, d), 6.76 (1H, dd), 3.08 (2H, t), 2.87 (2H, t).

### Example 34

¹H NMR (CD₃OD, 500 MHz) δ 8.01 (1H, d), 7.62 (1H, dd), 7.28 (4H, s), 7.12 (1H, d), 6.88 (1H, d), 6.75 (1H, dd), 3.06 (2H, t), 2.79 (2H, t).

### Example 35

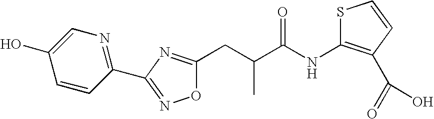

To a solution of ethyl 2-methyl-4-pentenoate (3.1 g) and NMO (6.4 g) in 20 mL of dichloromethane, was added OsO₄ (2.7 mL, 4% in water). After 12 h, to the mixture were added water (100 mL), dichloromethane (200 mL), and 30% isopropanol in chloroform (100 mL). The organic layer was concentrated. To the residue was added acetone and sodium periodate (9.3 g) in 50 mL of water. The white precipitate was formed and the slurry was stirred for 30 min and filtered. The filtrate was concentrated and extracted with dichloromethane (200 mL). The organic layer was dried with sodium sulfate and concentrated. The residue was purified by Biotage to give the aldehyde as a colorless oil. To this oil was added 15 mL of t-butanol, 2-methylbutene (10 mL), and a solution of sodium dihydrophosphate (12 g) and sodium chlorite (9 g, 80%) in 50 mL of water. After 1.5 h, the mixture was basified with NaOH. The organic layer was removed and the aqueous layer was acidified with HCl until pH=3. The mixture was extracted with ethyl acetate. The organic layer was dried with sodium sulfate and concentrated to give the monoacid as a dark oil. To this monoacid (7.2 g) in 45 mL of dichloromethane was added DMF (0.05 mL) and oxalylchloride (45 mL, 2N in dichloromethane) at 0° C. The mixture was stirred at 0° C. for 15 min and then rt for 1 h. The volatile was removed the residue was then treated with 17 (12.3 g) and 60 mL of pyridine. The resulting mixture was heated at 130° C. for overnight and pyridine was removed in vacuo. The residue was partitioned between water and dichloromethane. The organic layer was concentrated and purified by biotage (20-40% ethyl acetate in hexane) to give the oxadiazole as a brown oil. To this ethyl ester (155 mg) were added 10 mL of THF:methanol:water (3:1:1) and 1N lithium hydroxide solution (4 mL). After 2 b, the mixture was D concentrated. To the aqueous residue was added HCl until pH=4. This mixture was extracted with 30% isopropanol in chloroform (20 mL). The combined organic layers were dried with sodium sulfate and concentrated in vacuo to give the acid as a brown oil. This acid intermediate was elaborated into EXAMPLE 35 using conditions described in the Examples above. ¹H NMR (acetone-d₆, 500 MHz) δ 11.4 (1H, s), 8.32 (1H, s), 7.94 (1H, d), 7.35 (1H, dd), 7.23 (1H, d), 6.93 (1H, d), 3.47 (2H, m), 3.24 (1H, q), 1.47 (3H, d); LCMS m/z 375 (M+1).

### Biological Assays

The activity of the compounds of the present invention regarding niacin receptor affinity and function can be evaluated using the following assays:

### ³H-Niacin Binding Assay:

1. Membrane: Membrane preps are stored in liquid nitrogen in:
20 mM HEPES, pH 7.4
0.1 mM EDTA

Thaw receptor membranes quickly and place on ice. Resuspend by pipetting up and down vigorously, pool all tubes, and mix well. Use clean human at 15 μg/well, clean mouse at 10 ug/well, dirty preps at 30 ug/well.
1a. (human): Dilute in Binding Buffer.
1b. (human+4% serum): Add 5.7% of 100% human serum stock (stored at −20° C.) for a final concentration of 4%. Dilute in Binding Buffer.
1c. (mouse): Dilute in Binding Buffer.
   2. Wash buffer and dilution buffer: Make 10 liters of ice-cold Binding Buffer:
20 mM HEPES, pH 7.4
1 mM MgCl₂
0.01% CHAPS (w/v)
use molecular grade or ddH₂O water
   3. [5,6-³H]-nicotinic acid: American Radiolabeled Chemicals, Inc. (cat #ART-689). Stock is ˜50 Ci/mmol, 1 mCi/ml, 1 ml total in ethanol→20 μM

Make an intermediate ³H-niacin working solution containing 7.5% EtOH and 0.25 μM tracer. 40 μL of this will be diluted into 200 μL total in each well→1.5% EtOH, 50 nM tracer final.

4. Unlabeled nicotinic acid:

Make 100 mM, 10 mM, and 80 μM stocks; store at −20° C. Dilute in DMSO.

### 5. Preparing Plates:

1) Aliquot manually into plates. All compounds are tested in duplicate. 10 mM unlabeled nicotinic acid must be included as a sample compound in each experiment.
2) Dilute the 10 mM compounds across the plate in 1:5 dilutions (8 μl:40 μl).
3) Add 195 μL binding buffer to all wells of Intermediate Plates to create working solutions (250 μM→0. There will be one Intermediate Plate for each Drug Plate.
4) Transfer 5 μL from Drug Plate to the Intermediate Plate. Mix 4-5 times.

### 6. Procedure:

1) Add 140 μL of appropriate diluted 19CD membrane to every well. There will be three plates for each drug plate: one human, one human+serum, one mouse.
2) Add 20 μL of compound from the appropriate intermediate plate
3) Add 40 μL of 0.25 μM ³H-nicotinic acid to all wells.
4) Seal plates, cover with aluminum foil, and shake at RT for 3-4 hours, speed 2, titer plate shaker.
5) Filter and wash with 8×200 μL ice-cold binding buffer. Be sure to rinse the apparatus with >1 liter of water after last plate.
6) Air dry overnight in hood (prop plate up so that air can flow through).
7) Seal the back of the plate
8) Add 40 μL Microscint-20 to each well.
9) Seal tops with sealer.
10) Count in Packard Topcount scintillation counter.
11) Upload data to calculation program, and also plot raw counts in Prism, determining that the graphs generated, and the IC₅₀ values agree.

The compounds of the invention generally have an IC₅₀ in the ³H-nicotinic acid competition binding assay within the range of 1 nM to about 25 μM.

### ³⁵S-GTPγS Binding Assay:

Membranes prepared from Chinese Hamster Ovary (CHO)—K1 cells stably expressing the niacin receptor or vector control (7 μg/assay) were diluted in assay buffer (100 mM HEPES, 100 mM NaCl and 10 mM MgCl₂ pH 7.4) in Wallac Scintistrip plates and pre-incubated with test compounds diluted in assay buffer containing 40 μM GDP (final [GDP] was 10 μM) for ˜10 minutes before addition of ³⁵S-GTPγS to 0.3 nM. To avoid potential compound precipitation, all compounds were first prepared in 100% DMSO and then diluted with assay buffer resulting in a final concentration of 3% DMSO in the assay. Binding was allowed to proceed for one hour before centrifuging the plates at 4000 rpm for 15 minutes at room temperature and subsequent counting in a TopCount scintillation counter. Non-linear regression analysis of the binding curves was performed in GraphPad Prism.

### Membrane Preparation

### Materials:

CHO-K1 cell culture medium: F-12 Kaighn's Modified Cell Culture Medium with 10% FBS, 2 mM L-Glutamine, 1 mM Sodium Pyruvate and 400 μg/ml G418

| | | |
|---|---|---|
| Membrane Scrape Buffer: | 20 | mM HEPES |
| | 10 | mM EDTA, pH 7.4 |
| Membrane Wash Buffer: | 20 | mM HEPES |
| | 0.1 | mM EDTA, pH 7.4 |

| | |
|---|---|
| Protease Inhibitor Cocktail: | P-8340, (Sigma, St. Louis, MO) |

### Procedure:

(Keep everything on ice throughout prep; buffers and plates of cells)
Aspirate cell culture media off the 15 cm² plates, rinse with 5 mL cold PBS and aspirate.
Add 5 ml Membrane Scrape Buffer and scrape cells. Transfer scrape into 50 mL centrifuge tube. Add 50 μL Protease Inhibitor Cocktail.
Spin at 20,000 rpm for 17 minutes at 4° C.
Aspirate off the supernatant and resuspend pellet in 30 mL Membrane Wash Buffer. Add 50 μL Protease Inhibitor Cocktail.
Spin at 20,000 rpm for 17 minutes at 4° C.
Aspirate the supernatant off the membrane pellet. The pellet may be frozen at −80° C. for later use or it can be used immediately.

### Assay

### Materials:

Guanosine 5′-diphosphate sodium salt (GDP, Sigma-Aldrich Catalog #87127)
Guanosine 5′-[γ³⁵S]thiotriphosphate, triethylammonium salt ([³⁵S]GTPγS, Amersham Biosciences Catalog #SJ1320, ˜1000 Ci/mmol)
96 well Scintiplates (Perkin-Elmer #1450-501)
Binding Buffer: 20 mM HEPES, pH 7.4
   100 mM NaCl
   10 mM MgCl₂
GDP Buffer: binding buffer plus GDP, ranging from 0.4 to 40 PM, make fresh before assay

### Procedure:

(total assay volume=100 μwell)
25 μL GDP buffer with or without compounds (final GDP 10 μM—so use 40 μM stock)
50 μL membrane in binding buffer (0.4 mg protein/mL)
25 μL [³⁵S]GTPγS in binding buffer. This is made by adding 5 μl [³⁵S]GTPγS stock into 10 mL binding buffer (This buffer has no GDP)
   Thaw compound plates to be screened (daughter plates with 5 μL compound @ 2 mM in 100% DMSO)
   Dilute the 2 mM compounds 1:50 with 245 μL GDP buffer to 40 μM in 2% DMSO. (Note: the concentration of GDP in the GDP buffer depends on the receptor and should be optimized to obtain maximal signal to noise; 40 μM).
   Thaw frozen membrane pellet on ice. (Note: they are really membranes at this point, the cells were broken in the hypotonic buffer without any salt during the membrane prep step, and most cellular proteins were washed away)
   Homogenize membranes briefly (few seconds—don't allow the membranes to warm up, so keep on ice between bursts of homogenization) until in suspension using a POLYTRON PT3100 (probe PT-DA 3007/2 at setting of 7000 rpm). Determine the membrane protein concentration by Bradford assay. Dilute membrane to a protein concentrations of 0.40 mg/ml in Binding Buffer. (Note: the final assay concentration is 20 μg/well).
   Add 25 μL compounds in GDP buffer per well to Scintiplate.
      Add 50 μL of membranes per well to Scintiplate.
   Pre-incubate for 5-10 minutes at room temperature. (cover plates with foil since compounds may be light sensitive)
   Add 25 μL of diluted [³⁵S]GTPγS. Incubate on shaker (Lab-Line model #1314, shake at setting of 4) for 60 minutes at room temperature. Cover the plates with foil since some compounds might be light sensitive.
   Assay is stopped by spinning plates sealed with plate covers at 2500 rpm for 20 minutes at 22° C.
   Read on TopCount NXT scintillation counter—35S protocol.

The compounds of the invention generally have an EC₅₀ in the functional in vitro GTPγS binding assay within the range of about less than 1 μM to as high as about 100 μM.

### Flushing Via Laser Doppler

Male C57Bl6 mice (˜25 g) are anesthetized using 10 mg/ml/kg Nembutal sodium. When antagonists are to be administered they are co-injected with the Nembutal anesthesia. After ten minutes the animal is placed under the laser and the ear is folded back to expose the ventral side. The laser is positioned in the center of the ear and focused to an intensity of 8.4-9.0 V (with is generally ˜4.5 cm above the ear). Data acquisition is initiated with a 15 by 15 image format, auto interval, 60 images and a 20 sec time delay with a medium resolution. Test compounds are administered following the 10th image via injection into the peritoneal space. Images 1-10 are considered the animal's-baseline and data is normalized to an average of the baseline mean intensities.

### Materials and Methods—Laser Doppler Primed PimII; Niacin (Sigma); Nembutal (Abbott Labs).

All patents, patent applications and publications that are cited herein are hereby incorporated by reference in their entirety. While certain preferred embodiments have been described herein in detail, numerous alternative embodiments are seen as falling within the scope of the invention.

1. A compound represented by formula I: 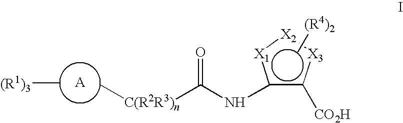
or a pharmaceutically acceptable salt or solvate thereof is disclosed wherein:
one of X¹, X² and X³ represents a sulfur atom, and the other two represent carbon or nitrogen atoms;
ring A represents a 6-10 membered aryl, or a 5-13 membered heteroaryl or partially aromatic heterocyclic group, said heteroaryl and partially aromatic heterocyclic group containing at least one heteroatom selected from O, S, S(O), S(O)₂ and N, and optionally containing 1 other heteroatom selected from O and S, and optionally containing 1-3 additional N atoms, with up to 5 heteroatoms being present;
each R² and R³ is independently H, C₁₋₃alkyl, haloC₁₋₃alkyl, OC₁₋₃alkyl, haloC₁₋₃alkoxy, OH or F;
n represents an integer of from 2 to 4;
each R⁴ is H or is independently selected from halo, SC₁₋₄alkyl, CN, C₁₋₄alkyl, C₁₋₄alkoxy, haloC₁₋₄alkyl and haloC₁₋₄alkoxy;
and each R¹ is H or is independently selected from the group consisting of:
a) halo, OH, CO₂H, CN, NH₂, S(O)₀₋₂R^{e}, C(O)R^{e}, OC(O)R^{e} and CO₂R^{e}, wherein R^{e} is C₁₋₄alkyl or phenyl, each being optionally substituted with 1-3 groups, 1-3 of which are halo or C₁₋₃alkyl, and 1-2 of which are selected from OC₁₋₃alkyl, haloC₁₋₃alkyl, haloC₁₋₃alkoxy, OH, NH₂ and NHC₁₋₃alkyl; b) C₁₋₆ alkyl and OC₁₋₆alkyl, said C₁₋₆alkyl and alkyl portion of OC₁₋₆alkyl being optionally substituted with 1-3 groups, 1-3 of which are halo and 1-2 of which are selected from: OH, CO₂H, CO₂C₁₋₄alkyl, CO₂C₁₋₄haloalkyl, OCO₂C₁₋₄alkyl, NH₂, NHC₁₋₄alkyl, N(C₁₋₄alkyl)₂, Hetcy and CN; c) NHC₁₋₄alkyl and N(C₁₋₄alkyl)₂, the alkyl portions of which are optionally substituted as set forth in (b) above;
d) C(O)NH₂, C(O)NHC₁₋₄alkyl, C(O)N(C₁₋₄alkyl)₂, C(O)Hetcy, C(O)NHOC₁₋₄alkyl and C(O)N(C₁₋₄alkyl)(OC₁₋₄alkyl), the alkyl portions of which are optionally substituted as set forth in (b) above;
e) NR′C(O)R″, NR′SO₂R″, NR′CO₂R″ and NR′C(O)NR″R′″ wherein:
R′ represents H, C₁₋₃alkyl or haloC₁₋₃alkyl,
R″ represents (a) C₁₋₈alkyl optionally substituted with 1-4 groups, 0-4 of which are halo, and 0-1 of which are selected from the group consisting of: OC₁₋₆alkyl, OH, CO₂H, CO₂C₁₋₄alkyl, CO₂C₁₋₄haloalkyl, NH₂, NHC₁₋₄alkyl, N(C₁₋₄alkyl)₂, CN, Hetcy, Aryl and HAR,
said Hetcy, Aryl and HAR being further optionally substituted with 1-3 halo, C₁₋₄alkyl, C₁₋₄alkoxy, haloC₁₋₄alkyl or haloC₁₋₄alkoxy groups;
(b) Hetcy, Aryl or HAR, each being optionally substituted with 1-3 members selected from the group consisting of: halo, C₁₋₄alkyl, C₁₋₄alkoxy, haloC₁₋₄alkyl and haloC₁₋₄alkoxy groups;
and R′″ representing H or R″;
f) phenyl or a 5-6 membered heteroaryl or a Hetcy group attached at any available ring atom and each being optionally substituted with 1-3 groups, 1-3 of which are selected from halo, C₁₋₃alkyl and haloC₁₋₃alkyl groups, and 1-2 of which are selected from OC₁₋₃alkyl and haloOC₁₋₃alkyl groups, and 0-1 of which is selected from the group consisting of:
i) OH; CO₂H; CN; NH₂ and S(O)₀₋₂R^{e} wherein R^{e} is as described above;
ii) NHC₁₋₄alkyl and N(C₁₋₄alkyl)₂, the alkyl portions of which are optionally substituted with 1-3 groups, 1-3 of which are halo and 1-2 of which are selected from: OH, CO₂H, CO₂C₁₋₄alkyl, CO₂C₁₋₄haloalkyl, NH₂, NHC₁₋₄alkyl, N(C₁₋₄alkyl)₂ and CN;
iii) C(O)NH₂, C(O)NHC₁₋₄alkyl, C(O)N(C₁₋₄alkyl)₂, C(O)NHOC₁₋₄alkyl and C(O)N(C₁₋₄alkyl)(OC₁₋₄alkyl), the alkyl portions of which are optionally substituted as set forth in b) above; and
iv) NR′C(O)R″, NR′SO₂R″, NR′CO₂R″ and NR′C(O)NR″R′″
wherein R′, R″ and R′″ are as described above.

2. (canceled)

3. A compound in accordance with claim 1 wherein: ring A is selected from the group consisting of: phenyl, naphthyl, isoxazolyl, isothiazolyl, pyrazolyl, pyridyl, oxazolyl, oxadiazolyl, thiadiazolyl, thiazolyl, triazolyl, thienyl, pyrimidyl, benzothiazolyl, or a member selected from the group consisting of:

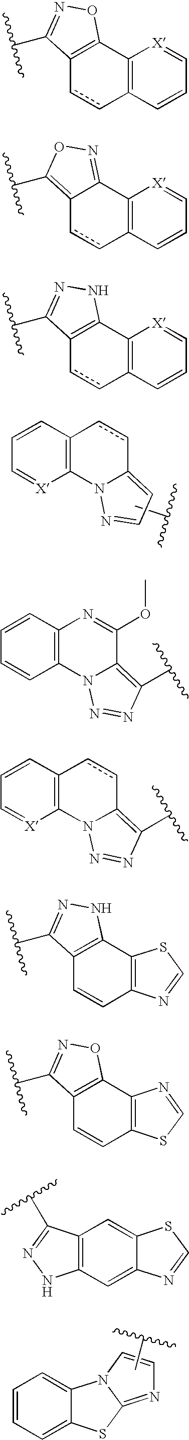

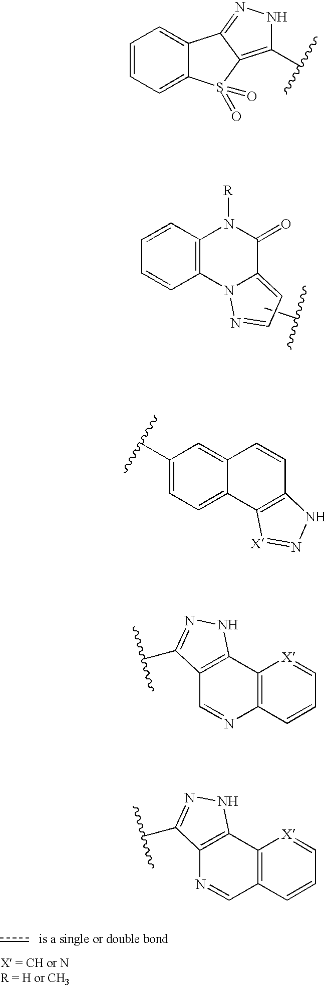

4. A compound in accordance with claim 3 wherein ring A is selected from the group consisting of: phenyl, naphthyl, isoxazolyl, pyrazolyl, oxazolyl, oxadiazolyl, thiazolyl, triazolyl, and benzothiazolyl.

5. A compound in accordance with claim 4 wherein ring A is selected from the group consisting of: phenyl, naphthyl and oxadiazolyl.

6. A compound in accordance with claim 1 wherein one of X¹, X² and X³ is S, one is C and one is C or N.

7. A compound in accordance with claim 6 wherein one of X¹, X² and X³ is S, and the other two are C.

8. A compound in accordance with claim 1 wherein each R¹ is H or is selected from the group consisting of:
a) halo, OH, CO₂H, CN, NH₂, S(O)₀₋₂R^{e}, C(O)R^{e}, OC(O)R^{e} and CO₂R^{e}, wherein R^{e} is C₁₋₄alkyl or phenyl, each being optionally substituted with 1-3 groups, 1-3 of which are halo or C₁₋₃alkyl, and 1-2 of which are selected from OC₁₋₃alkyl, haloC₁₋₃alkyl, haloC₁₋₃alkoxy, OH, NH₂ and NHC₁₋₃alkyl;
b) C₁₋₆ alkyl and OC₁₋₆alkyl, said C₁₋₆alkyl and alkyl portion of OC₁₋₆alkyl being optionally substituted with 1-3 groups, 1-3 of which are halo and 1-2 of which are selected from: OH, CO₂H, CO₂C₁₋₄alkyl, CO₂C₁₋₄haloalkyl, OCO₂C₁₋₄alkyl, NH₂, NHC₁₋₄alkyl, N(C₁₋₄alkyl)₂, Hetcy and CN; and
c) phenyl or a 5-6 membered heteroaryl or a Hetcy group attached at any available ring atom and each being optionally substituted with 1-3 groups, 1-3 of which are selected from halo, C₁₋₃alkyl and haloC₁₋₃alkyl groups, and 1-2 of which are selected from OC₁₋₃alkyl and haloOC₁₋₃alkyl groups, and 0-1 of which is selected from the group consisting of:
i) OH; CO₂H; CN; NH₂ and S(O)₀₋₂R^{e} wherein R^{e} is as described above;
ii) NHC₁₋₄alkyl and N(C₁₋₄alkyl)₂, the alkyl portions of which are optionally substituted with 1-3 groups, 1-3 of which are halo and 1-2 of which are selected from: OH, CO₂H, CO₂C₁₋₄alkyl, CO₂C₁₋₄haloalkyl, NH₂, NHC₁₋₄alkyl, N(C₁₋₄alkyl)₂ and CN;
iii) C(O)NH₂, C(O)NHC₁₋₄alkyl, C(O)N(C₁₋₄alkyl)₂, C(O)NHOC₁₋₄alkyl and C(O)N(C₁₋₄alkyl)(OC₁₋₄alkyl), the alkyl portions of which are optionally substituted as set forth in b) above; and
iv) NR′C(O)R″, NR′SO₂R″, NR′CO₂R″ and NR′C(O)NR″R′″ wherein R′, R″ and R′″ are as described above with respect to formula I.

9. A compound in accordance with claim 8 wherein each R¹ is H or is selected from the group consisting of:
a) halo or OH;
b) C₁₋₄alkyl and OC₁₋₄alkyl, each optionally substituted with 1-3 halo groups;
c) phenyl or a 5-6 membered heteroaryl group optionally substituted with 1-3 groups, 1-3 of which are selected from halo, C₁₋₃alkyl and haloC₁₋₃alkyl groups, and 1-2 of which are selected from OC₁₋₃alkyl and haloOC₁₋₃alkyl groups, and 0-1 of which is OH.

10. (canceled)

11. A compound in accordance with claim 1 wherein R² and R³ are independently H, C₁₋₃alkyl or haloC₁₋₃alkyl.

12. (canceled)

13. A compound in accordance with claim 1 wherein n represents the integer 2 or 4.

14. (canceled)

15. (canceled)

16. A compound in accordance with claim 1 wherein each R⁴ is H or is independently selected from halo, C₁₋₄alkyl, CN and SC₁₋₄alkyl.

17. (canceled)

18. A compound in accordance with claim 1 wherein:
ring A is a phenyl or naphthyl group, or a 5-6 membered monocyclic heteroaryl group
one of X¹, X² and X³ is S, one is C and one is C or N;
each R¹ is H or is selected from the group consisting of:
a) halo, OH, CN, NH₂, S(O)₀₋₂R^{e}, C(O)R^{e}, OC(O)R^{e} and CO₂R^{e}, wherein R^{e} is C₁₋₄alkyl or phenyl, each being optionally substituted with 1-3 groups, 1-3 of which are halo or C₁₋₃alkyl, and 1-2 of which are selected from OC₁₋₃alkyl, haloC₁₋₃alkyl, haloC₁₋₃alkoxy, OH, NH₂ and NHC₁₋₃alkyl;
b) C₁₋₆ alkyl and OC₁₋₆alkyl, said C₁₋₆alkyl and alkyl portion of OC₁₋₆alkyl being optionally substituted with 1-3 groups, 1-3 of which are halo and 1-2 of which are selected from: OH, CO₂H, CO₂C₁₋₄alkyl, CO₂C₁₋₄haloalkyl, OCO₂C₁₋₄alkyl, NH₂, NHC₁₋₄alkyl, N(C₁₋₄alkyl)₂, Hetcy and CN; and
c) phenyl or a 5-6 membered heteroaryl or a Hetcy group attached at any available ring atom and each being optionally substituted with 1-3 groups, 1-3 of which are selected from halo, C₁₋₃alkyl and haloC₁₋₃alkyl groups, and 1-2 of which are selected from OC₁₋₃alkyl and haloOC₁₋₃alkyl groups, and 0-1 of which is selected from the group consisting of:
i) OH, CN, and NH₂;
ii) C(O)NH₂, C(O)NHC₁₋₄alkyl, C(O)N(C₁₋₄alkyl)₂, C(O)NHOC₁₋₄alkyl and C(O)N(C₁₋₄alkyl)(OC₁₋₄alkyl), the alkyl portions of which are optionally substituted as set forth in b) above; and
iii) NR′C(O)R″, NR′SO₂R″, NR′CO₂R″ and NR′C(O)NR″R′″ wherein R′, R″ and R′″ are as described above with respect to formula I;
R² and R³ are independently H or C₁₋₃alkyl;
n represents the integer 2 or 4; and
R⁴ is H or is independently selected from halo, C₁₋₄alkyl, CN and SC₁₋₄alkyl.

19. A compound in accordance with claim 1 selected from the following table:
TABLE

| | |
|---|---|
| EXAMPLE 1 | EXAMPLE 2 |
| 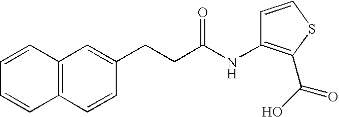 | 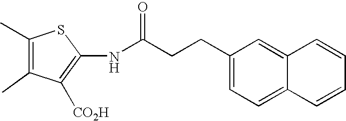 |
| EXAMPLE 3 | EXAMPLE 4 |
| 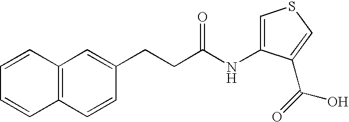 | 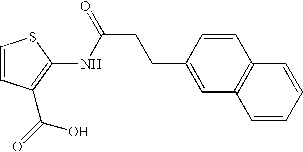 |
| EXAMPLE 5 | EXAMPLE 6 |
| 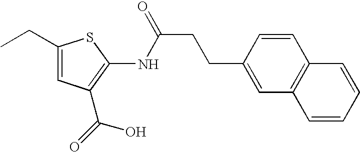 | 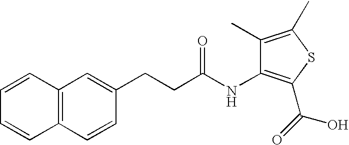 |
| EXAMPLE 7 | EXAMPLE 8 |
| 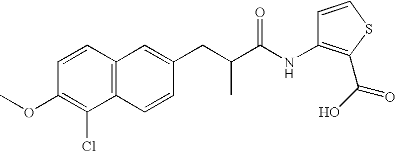 | 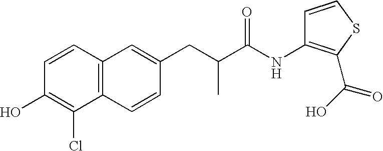 |
| EXAMPLE 9 | EXAMPLE 10 |
| 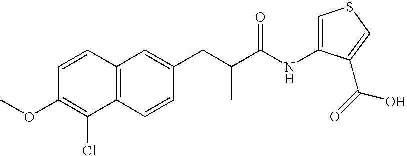 | 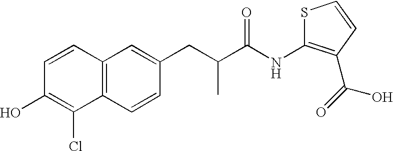 |
| EXAMPLE 11 | EXAMPLE 12 |
| 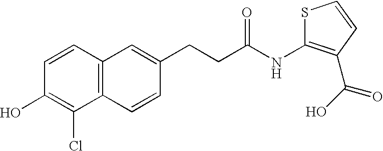 | 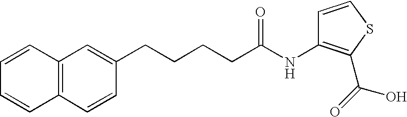 |
| EXAMPLE 13 | EXAMPLE 14 |
| 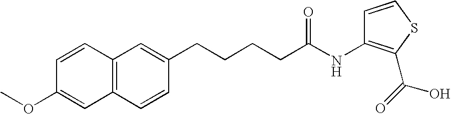 | 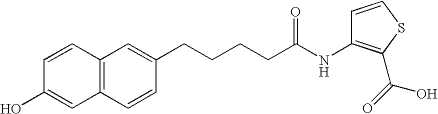 |
| EXAMPLE 15 | EXAMPLE 16 |
| 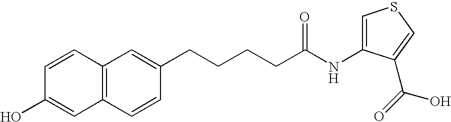 | 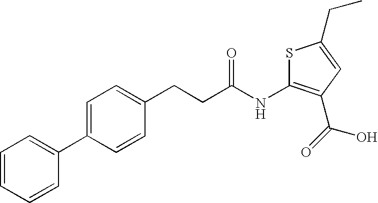 |
| EXAMPLE 17 | EXAMPLE 18 |
| 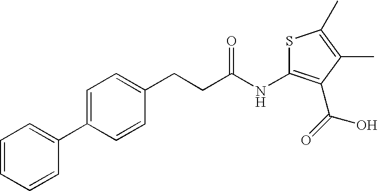 | 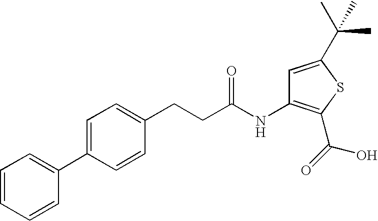 |
| EXAMPLE 19 | EXAMPLE 20 |
| 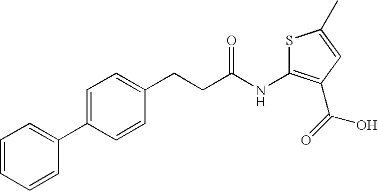 | 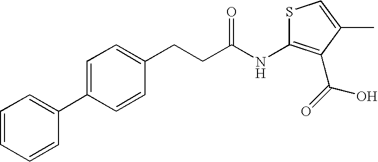 |
| EXAMPLE 21 | EXAMPLE 22 |
| 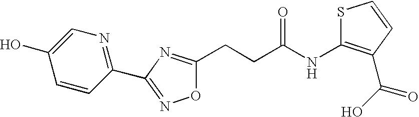 | 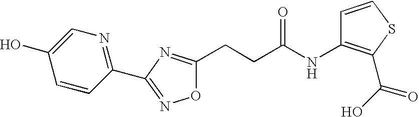 |
| EXAMPLE 23 | EXAMPLE 24 |
| 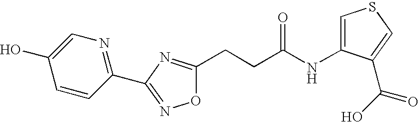 | 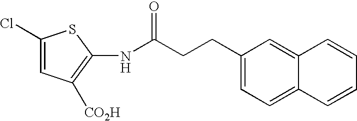 |
| EXAMPLE 25 | EXAMPLE 26 |
| 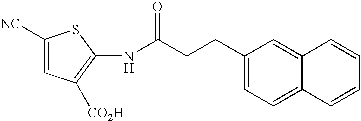 | 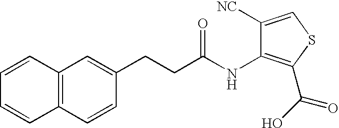 |
| EXAMPLE 27 | EXAMPLE 28 |
| 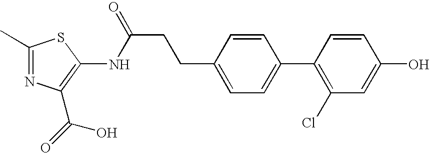 | 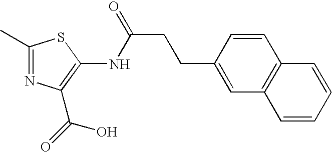 |
| EXAMPLE 29 | EXAMPLE 30 |
| 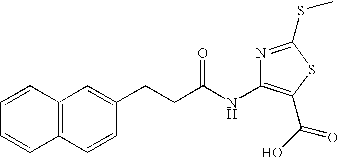 | 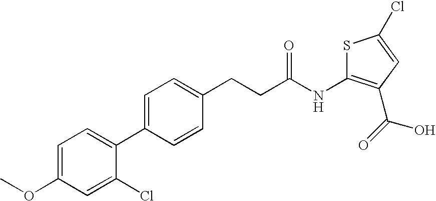 |
| EXAMPLE 31 | EXAMPLE 32 |
| 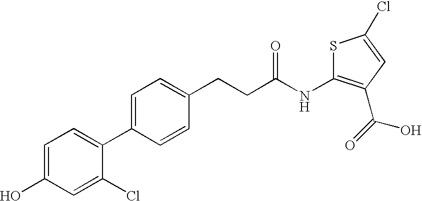 | 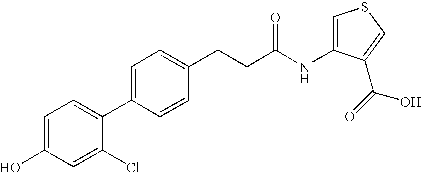 |
| EXAMPLE 33 | EXAMPLE 34 |
| 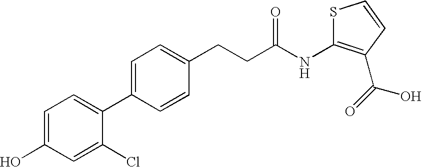 | 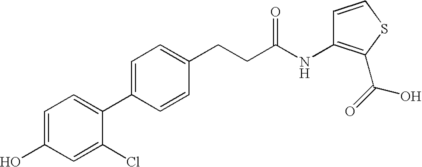 |

EXAMPLE 35 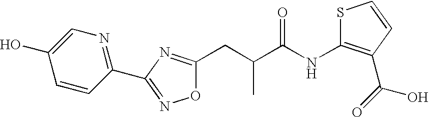
or a pharmaceutically acceptable salt or solvate thereof.

20. A pharmaceutical composition comprising a compound in accordance with claim 1 in combination with a pharmaceutically acceptable carrier.

21. A method of treating atherosclerosis in a human patient in need of such treatment comprising administering to the patient a compound of claim 1 in an amount that is effective for treating atherosclerosis.

22. A method of treating dyslipidemia in a human patient in need of such treatment comprising administering to the patient a compound of claim 1 in an amount that is effective for treating dyslipidemias.

23. A method of treating diabetes in a human patient in need of such treatment comprising administering to the patient a compound of claim 1 in an amount that is effective for treating diabetes.

24. A method of treating metabolic syndrome in a human patient in need of such treatment comprising administering to the patient a compound of claim 1 in an amount that is effective for treating metabolic syndrome.

25. A method of treating atherosclerosis, dyslipidemias, diabetes, metabolic syndrome or a related condition in a human patient in need of such treatment, comprising administering to the patient a compound of claim 1 and a DP receptor antagonist, said compounds being administered in an amount that is effective to treat atherosclerosis, dyslipidemia, diabetes or a related condition in the absence of substantial flushing.

26. A method in accordance with claim 21 wherein the DP receptor antagonist selected from the group consisting of compounds A through AJ: 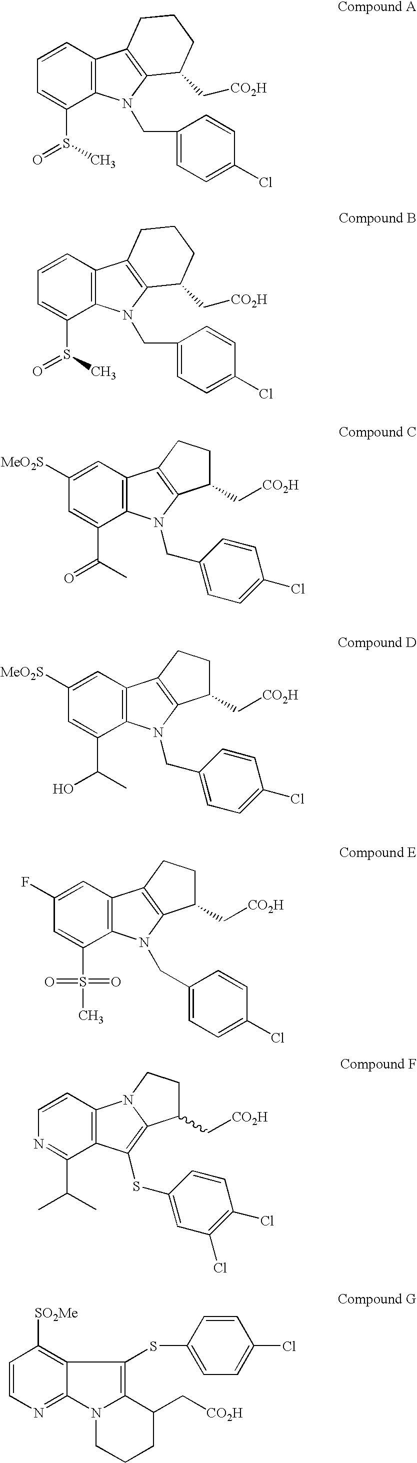 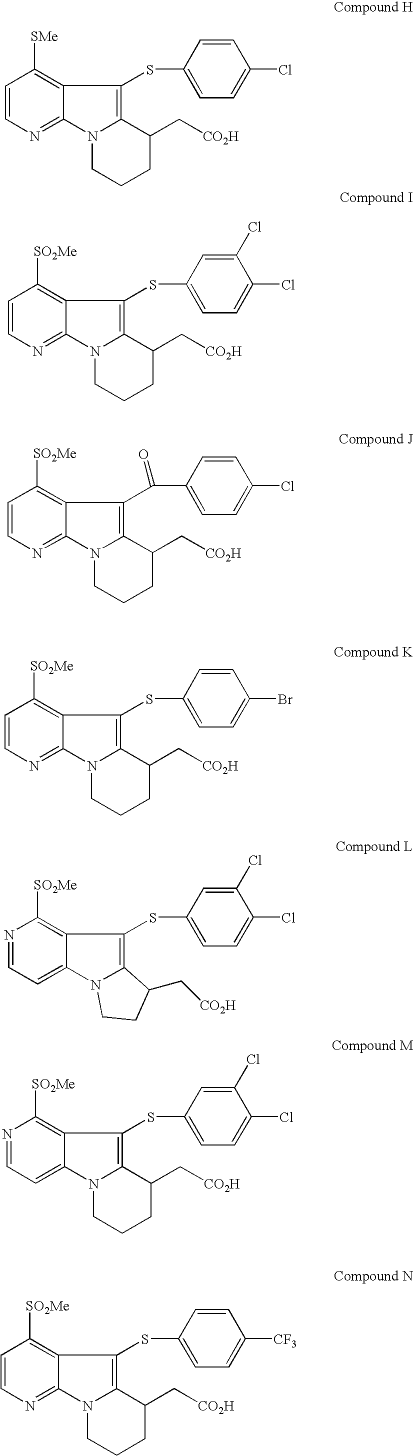 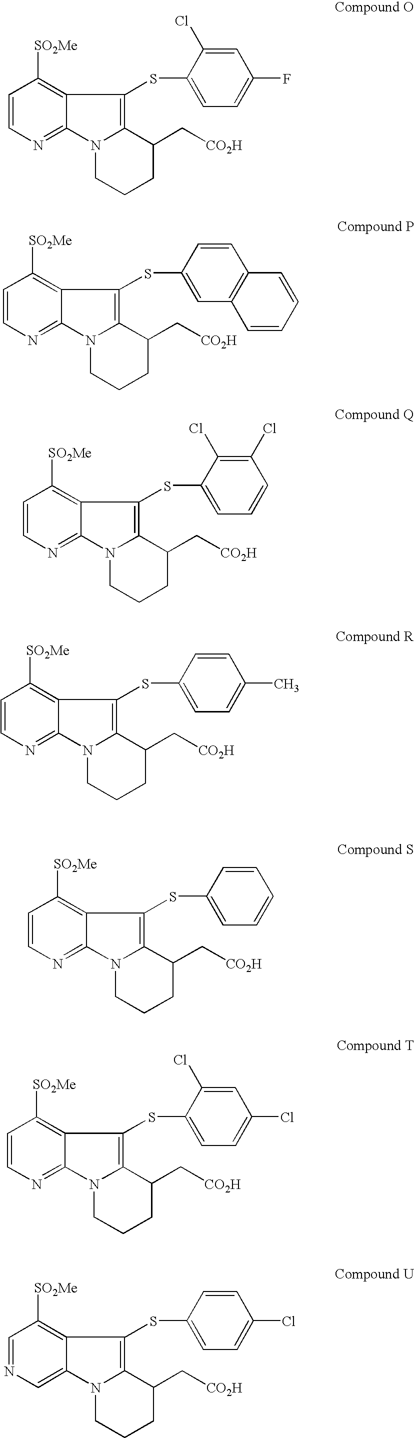 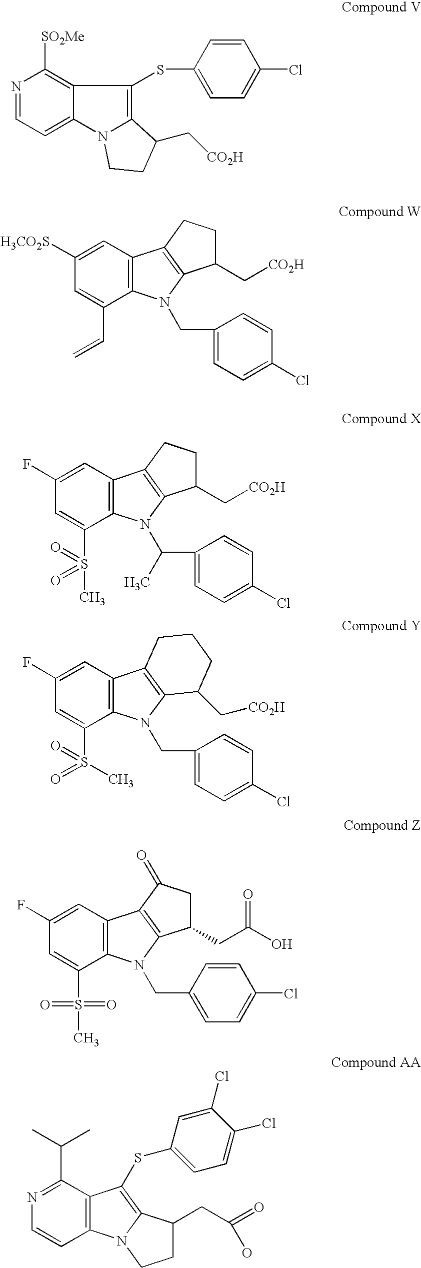 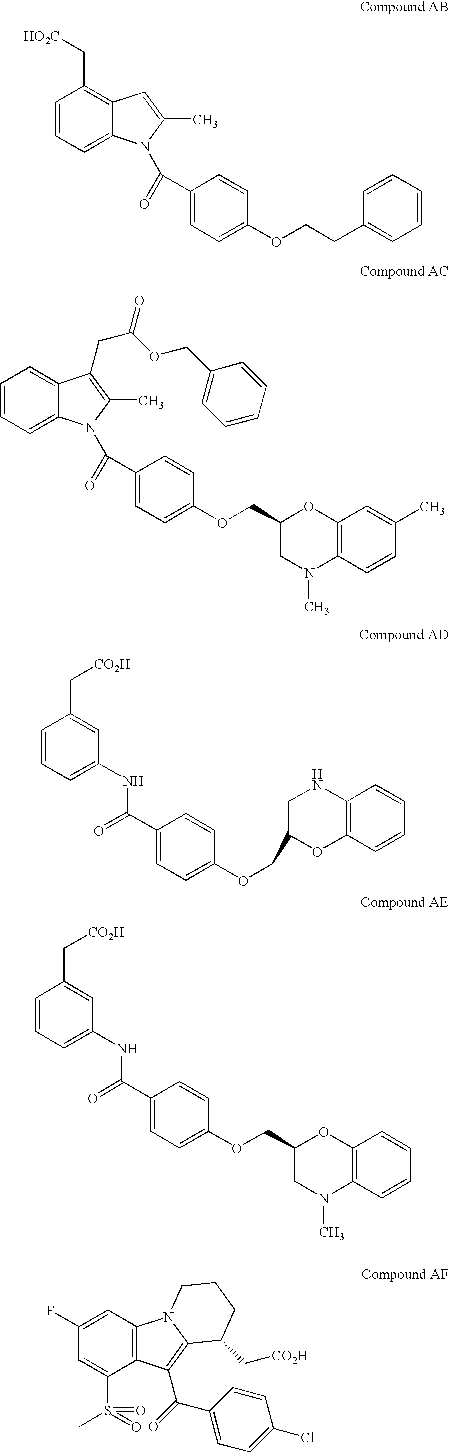 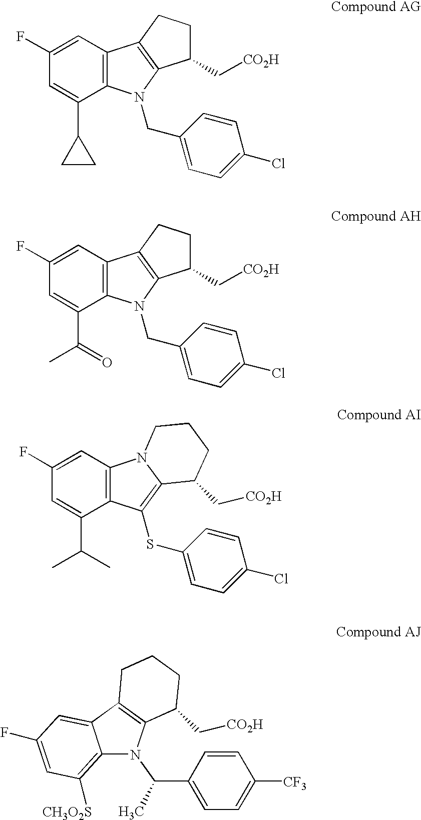
or a pharmaceutically acceptable salt or solvate thereof.
